(19) United States
(12) Patent Application Publication
(10) Pub. No.: US 2023/0079117 A1
(43) Pub. Date: Mar. 16, 2023

(54) DEVICES, SYSTEMS, AND METHODS FOR FLUID DELIVERY

(71) Applicant: Certus Critical Care, Inc., Salt Lake City, UT (US)

(72) Inventors: Ruth BEEBY, Santa Clara, CA (US); Patrick Ryan KOLBAY, Salt Lake City, UT (US); David POISNER, Carmichael, CA (US); Daniel FONG, Sacramento, CA (US)

(21) Appl. No.: 17/842,729

(22) Filed: Jun. 16, 2022

Related U.S. Application Data
(60) Provisional application No. 63/244,719, filed on Sep. 15, 2021.

(51) Int. Cl. A61M 25/10; A61B 17/12

(52) U.S. Cl. CPC A61M 25/10182; A61B 17/12136; A61B 17/12109; A61M 5/14546

(57) ABSTRACT

Devices, systems, and methods for minimization of backlash in a syringe pump are described herein. Syringe pumps may comprise a syringe including a cylindrical body and a plunger, and a controller. The plunger may comprise a linear gear that may operably couple with a circular gear in the controller. The controller may be configured to bias the circular gear in a biasing rotational direction, thereby positioning the plunger to move towards a biasing translational direction. The methods may comprise determining a backlash compensation amount, determining a fluid transfer movement amount, moving at least one component of the syringe pump the backlash compensation amount and the fluid transfer movement amount in a first direction, and moving the at least one component of the syringe pump a biasing movement amount in a second direction opposite the first direction to re-engage the first gear and the second gear to minimize backlash.

### CROSS REFERENCE TO RELATED APPLICATIONS

This application claims priority to U.S. provisional patent application number 63/244,719, filed on Sep. 15, 2021, the contents of which is incorporated herein by reference in its entirety.

### GOVERNMENT SUPPORT

This invention was made with government support under grant FA8650-20-2-6116 and W81XWH-21-C-0058 awarded by the United States Air Force/Air Force Material Command. The government has certain rights in the invention.

### FIELD

This application generally relates to devices, systems, and methods for the controlled delivery of fluids. The fluids may be delivered by syringe pumps configured to precisely meter fluids.

### BACKGROUND

Syringe pumps are ubiquitous in the biomedical field and in hospitals and are often used to deliver small doses of fluids such as blood products, intravenous fluids, and medications. They are also used during medical procedures such as endovascular procedures. For instance, syringe pumps have been used to deliver and/or withdraw fluid from balloon catheters during endovascular procedures to control blood flow in patients. Traditional syringe pumps are manual. A user may manually control the syringe pump to deliver and/or withdraw fluid from the syringe pump. However, relying on manual control may make it challenging to precisely control the flow rate of fluid and amount of fluid that may be transferred to and from the syringe pumps.

More recently, some syringe pumps utilizing lead screws have been developed for more automated control of fluid transfer. These syringe pumps, however, are typically heavy and bulky, and often require significant maintenance, thus making them challenging to use. Moreover, lead-screw based actuation typically utilizes lead screws that are separate from the syringe itself, and often longer than the plunger, thus requiring additional components and increasing the footprint of the pump.

Accordingly, there is a need for new and useful devices for precisely delivering fluid.

### SUMMARY

Devices, systems, and methods for fluid delivery are described herein. In some variations, a method of operating a syringe pump comprising a syringe, a processor communicably coupled to the syringe, a first gear and a second gear may comprise determining a backlash compensation amount and a fluid transfer movement amount via the processor. The method may include moving at least one component of the syringe pump the backlash compensation amount and the fluid transfer movement amount in a first direction via the processor. The method may include moving the at least one component of the syringe pump a biasing movement amount in a second direction opposite the first direction to re-engage the first gear and the second gear via the processor.

In some variations, an absolute value of the biasing movement amount may be equal to an absolute value of the backlash compensation amount. In some variations, moving the at least one component may comprise moving a motor operably coupled to the first gear. In some variations, moving the at least one component may comprise moving the first gear. In some variations, the first gear may be a circular gear and the second gear may be a linear gear. A plunger of the syringe may comprise the linear gear.

In some variations, the syringe pump may comprise a gearbox operably coupling a motor to the first gear. The backlash compensation amount may be based at least in part on a gear ratio of the gearbox. The gear ratio of the gearbox may be between about 40:1 and about 150:1. A module for the gearbox may be between about 0.4 mm and about 1.0 mm.

In some variations, the method may further comprise receiving a pressure measurement indicative of a pressure inside the syringe at the processor. The backlash compensation amount may be based at least in part on the pressure measurement.

In some variations, the first direction may be a fluid withdrawal direction and the second direction may be a fluid injection direction. In some variations, a blood flow control device may be fluidly coupled to the syringe pump. The method may further comprise advancing a distal portion of the blood flow control device to a blood vessel of a subject. The advancement may occur before determining the backlash compensation amount. The blood flow control device may communicatively coupled to a system controller. The system controller may include the processor.

In some variations, the method may further comprise determining a sum of the backlash compensation amount and the fluid transfer movement amount. The sum may be determined before moving the at least one component in the first direction. In some variations, moving the at least one component in the first direction may cause fluid transfer. In some variations, the syringe pump may comprise a plunger including a linear gear, and a controller comprising a circular gear. The circular gear may be operably coupled to the linear gear. Moving the at least one component in the second direction may re-engage the linear gear with the circular gear without materially moving the plunger.

In some variations, a syringe pump may comprise a cylindrical body, a plunger partially disposed within the cylindrical body. The plunger may comprise a first gear. The syringe pump may also comprise a controller comprising a second gear operably coupled to the first gear. The controller may be configured to: determine a backlash compensation amount, determine a fluid transfer movement amount, move at least one component of the syringe pump the backlash compensation amount and the fluid transfer movement amount in a first direction, and move the at least one component of the syringe pump a biasing movement amount in a second direction opposite the first direction to re-engage the first gear and the second gear.

In some variations, an absolute value of the biasing movement amount may be equal to an absolute value of the backlash compensation amount. In some variations, the controller may further comprise a motor operably coupled to the second gear to actuate the second gear. The at least one component may include the motor. In some variations, the first gear may be a linear gear and the second gear may be a circular gear. The at least one component may include the circular gear.

In some variations, the controller may further comprise a gearbox operably coupled to the second gear. The backlash compensation amount may be based at least in part on a gear ratio of the gearbox. The gear ratio of the gearbox may be between about 40:1 and about 150:1. A module for the gearbox may be between about 0.4 mm and about 1.0 mm.

In some variations, the controller may further comprise a pressure sensor to receive a pressure measurement indicative of a pressure inside the cylindrical body. The backlash compensation amount may be based at least in part on the pressure inside the cylindrical body. In some variations, the first direction may be a fluid withdrawal direction and the second direction may be a fluid injection direction.

In some variations, a blood flow control system may comprise the syringe pump and a blood flow control device fluidly coupled to the syringe pump. In some variations, the controller may be a system controller for the blood flow control system.

In some variations, a syringe pump may comprise a cylindrical body, a plunger partially disposed within the cylindrical body, and a controller. The plunger may comprise a linear gear. The controller may comprise a circular gear operably coupled to the linear gear, a motor operably coupled to the circular gear to actuate the circular gear, and a processor communicably coupled to the motor. The processor may be configured to bias the circular gear in a biasing rotational direction after the syringe pump transfers fluid.

In some variations, biasing the circular gear in the biasing rotational direction may position the plunger to move in a biasing translational direction. The syringe pump may be configured such that the plunger may remain substantially stationary when the circular gear is biased in the biasing rotational direction. In some variations, the biasing rotational direction may be a clockwise direction. In some variations, the biasing rotational direction may a counterclockwise direction.

In some variations, the processor may be configured to automatically bias the circular gear after the syringe pump receives fluid. In some variations, the processor may be configured to automatically bias the circular gear after the syringe pump delivers fluid. In some variations, the controller may further comprise a gearbox operably coupling the motor to the circular gear.

In some variations, a method may comprise advancing an expandable member of a blood flow control device to a target location in a blood vessel of a patient. The expandable member may be fluidly coupled to a syringe pump. The method may also comprise in response to determining that the expandable member is to be deflated, moving a component of the syringe pump in a first direction to compensate for backlash in the syringe pump and to deflate the expandable member, and after moving the component in the first direction, automatically moving the component in a second opposite direction.

In some variations, moving the component of the syringe pump in the first direction may deflate the expandable member. In some variations, the syringe pump may comprise a motor operably coupled to a circular gear. In some variations, the component may be the motor. The first direction may be a counterclockwise direction and the second direction may be a clockwise direction. In some variations, the component may be the motor, and the first direction may be a clockwise direction and the second direction may be a counterclockwise direction. In some variations, the component may be the circular gear, and the first direction may be a counterclockwise direction and the second direction may be a clockwise direction.

In some variations, the syringe pump may comprise a plunger, and moving the component in the first direction may cause fluid transfer. In some variations, the syringe pump may comprise a plunger including a linear gear, and a controller comprising a circular gear. The circular gear may be operably coupled to the linear gear. Moving the component in the second opposite direction may re-engage the linear gear with the circular gear without materially moving the plunger.

In some variations, a syringe pump may comprise a cylindrical body, a plunger partially disposed within the cylindrical body, and a controller. The plunger may comprise a linear gear. The controller may comprise a circular gear operably coupled to the linear gear, a motor operably coupled to the circular gear to actuate the circular gear, and a processor communicably coupled to the motor and configured to: in response to determining that an expandable member of a blood flow control device fluidly coupled to the syringe pump is to be deflated, move a component of the syringe pump in a first direction to compensate for backlash in the syringe pump and to deflate the expandable member, and after moving the component in the first direction, automatically move the component in a second opposite direction to re-engage the linear gear with the circular gear.

In some variations, the component may be the motor, and the first direction may be a counterclockwise direction and the second direction may be a clockwise direction. In some variations, the component may be the circular gear, and the first direction may be a counterclockwise direction and the second opposite direction may be a clockwise direction. In some variations, moving the component in the first direction may cause the fluid transfer. In some variations, moving the component in the second opposite direction may cause the plunger to remain substantially stationary.

In some variations, a syringe pump may comprise a cylindrical body, a plunger partially disposed within the cylindrical body, and a controller. The plunger may comprise a linear gear. The controller may a circular gear operably coupled to the linear gear, a motor operably coupled to the circular gear and configured to actuate the circular gear, and a processor communicably coupled to the motor and configured to: in response to determining that the plunger is to be moved in a first linear direction, rotate the circular gear in a first rotational direction, thereby moving the plunger in the first linear direction, and automatically rotate the circular gear in a second opposite rotational direction by a determined amount. Rotating the circular gear in the second rotational direction re-engages teeth of the linear gear with teeth of the circular gear thereby preparing the plunger to be moved in a second, opposite linear direction.

In some variations, the determined amount may be determined based on a lookup table. In some variations, the pump controller may further comprise a gearbox operably coupling the motor to the circular gear. In some variations, the determined amount may be based at least in part on a gear ratio of the gearbox. In some variations, a gear ratio of the gearbox may be between about 40:1 and 150:1. In some variations, the gear ratio may be about 90:1. In some variations, a module may be between about 0.4 mm and about 1.0 mm. In some variations, the module may be about 0.4 mm.

In some variations, the motor may be a stepper motor, and the determined amount may be at least in part on a step angle of the stepper motor. In some variations, the determined amount may be based at least in part on a gear ratio between the circular gear and the linear gear. In some variations, the determined amount may be calculated based on an amount of backlash compensation between the teeth of the linear gear and the teeth of the circular gear. In some variations, the pump controller may further comprise a gearbox operably coupling the motor to the circular gear, and the determined amount may be calculated based at least in part on a backlash compensation value between teeth of a first gear in the gearbox and teeth of a second gear in the gearbox. In some variations, the pump controller may be further coupled to an expandable member, and the determined amount is calculated based at least in part on a pressure value in the expandable member.

In some variations, at least one of the plunger and the pump controller may further comprise a sensor configured to track a position of the motor. In some variations, the determined amount may be calculated based on position of the motor. In some variations, at least one of the plunger and the pump controller may further comprise an optical sensor configured to track a movement of the plunger. In some variations, the sensor may comprise a contact sensing linear potentiometer, the plunger may include a plurality of fiducial markers, and the contact sensing linear potentiometer may measure a position of the plunger based on an engagement with at least one fiducial marker of the plurality of fiducial markers. In some variations, a distance between adjacent fiducial markers of the plurality of fiducial markers may be unique.

In some variations, the syringe pump may be fluidly coupled to an expandable member. In some variations, an expandable member pressure sensor may be coupled to the controller to measure pressure inside the expandable member, and the determined amount may be calculated based on a spike in a pressure data obtained from the expandable member pressure sensor. In some variations, the expandable member pressure sensor may be communicably coupled to a system controller. The system controller may be configured to: receive the pressure data from the pressure sensor, determine a rate of change in the received pressure values from the pressure data, and compare the rate of change with a threshold value to determine if the plunger has moved.

In some variations, the expandable member pressure sensor may be communicably coupled to a system controller. The system controller may be configured to receive the pressure data from the pressure sensor, determine a duration of a change in the received pressure values from the pressure data, and compare the duration of the change with a threshold value to determine if the plunger has moved.

In some variations, the pump controller may further comprise a motor current measurement unit configured to measure a motor current. In some variations, the determined amount may be calculated based on the measured motor current. In some variations, the pump controller may further be configured to compare a rate of change in the motor current with a threshold value to determine if the plunger has moved. In some variations, the syringe pump may be in fluid communication with an expandable member of a blood flow control device and may be configured to change a volume of the expandable member. In some variations, the syringe pump may be configured to deliver medication to a patient. In some variations, a blood flow control system may comprise a blood flow control device, and the syringe pump.

In some variations, a method of operating a syringe pump may comprise determining a desired movement for a motor of the syringe pump via a processor. The desired movement may be in a first rotational direction and the syringe pump may further comprises a plunger comprising a linear gear and a pump controller. The pump controller may comprise a circular gear operably coupled to the linear gear, the motor, and the processor, the motor is operably coupled to the circular gear and configured to actuate the circular gear, and the processor is communicably coupled to the motor. In some variations, the method may also comprise in response to determining that the first rotational direction corresponds with a withdrawing action of the plunger, determining, via the processor, a backlash movement for the motor, wherein the backlash movement is in a second, opposite rotational direction, determining, via the processor, a total movement for the motor in the first rotational direction. The total movement of the motor may be a sum of the desired movement and the backlash movement in the first rotational direction. The method may also comprise rotating the motor in the first rotational direction the total movement and rotating the motor in the second rotational direction the backlash movement.

In some variations, the desired movement may be calculated based on a desired volume of fluid to be delivered. In some variations, the method may further comprise in response to determining that the first rotational direction corresponds with an injection action of the plunger, rotating the motor in the first rotational direction based on the desired movement. In some variations, the motor may be operably coupled to the circular gear via a gearbox.

In some variations, a syringe pump configured to change a volume of an expandable member of a blood flow control device may comprise a cylindrical body, a plunger comprising an enlarged first end slidably positioned within the cylindrical body and an elongate member extending from the enlarged first end. The elongate member may comprise a plurality of fiducial markers configured to engage with a potentiometer to track a position of the plunger and a linear gear. The linear gear may be configured to be engaged by a circular gear such that the movement of the circular gear actuates the plunger.

In some variations, the plurality of fiducial markers may be non-linearly spaced such that at most two fiducial markers of the plurality of fiducial markers engage with the potentiometer at a time. In some variations, the plurality of fiducial markers may comprise a set of fiducial marker pairs. A distance between each fiducial marker in each of the set of fiducial marker pairs may be smaller than a distance between a fiducial marker pair of the set of fiducial marker pairs and an adjacent fiducial marker pair of the set of fiducial marker pairs.

In some variations, a system may comprise the syringe pump and a controller releasably coupled to the syringe pump. The controller may comprise a controller housing comprising the potentiometer. In some variations, a voltage divider may be created upon the engagement of the potentiometer with at least one fiducial marker of the plurality of fiducial markers. In some variations, a voltage measured from the voltage divider may be indicative of the position of the plunger. In some variations, the plurality of fiducial markers may be spaced asymmetrically on a plurality of parallel tracks. In some variations, the potentiometer may include a plurality of tracks configured to engage with the plurality of fiducial markers. In some variations, the engagement of the potentiometer with at least one fiducial marker may generate a unique voltage pattern indicative of the position of the plunger.

In some variations, a method may comprise advancing a device comprising a syringe pump to a target location in a blood vessel of a patient, rotating a circular gear of the syringe pump in a first rotational direction via a controller. Rotating the circular gear in the first rotational direction may translate a linear gear of the syringe pump in a first translational direction. The method may comprise automatically rotating the circular gear in the first rotational direction by a determined amount. Rotating the circular gear in the first rotational direction may re-engage teeth of the linear gear with teeth of the circular gear. In some variations, the device may be a blood flow control device. In some variations, the controller may be a device controller. In some variations, the controller may be a pump controller.

### BRIEF DESCRIPTION OF THE DRAWINGS

FIG. 1 illustrates an exemplary variation of a syringe.

FIGS. 2A-2C depict an exemplary variation of a syringe pump. FIG. 2A shows the syringe coupled to the pump housing; FIG. 2B shows the housing alone; and FIG. 2C provides a cross-sectional view of the syringe pump that shows more details of a circular gear and a linear gear.

FIGS. 3A illustrates an exemplary variation of fiducial markers along a plunger of a syringe of the blood flow control device.

FIG. 3B illustrates an exemplary variation of an electrical circuit created when one fiducial marker is engaged with a linear potentiometer.

FIG. 3C illustrates an exemplary variation of an electrical circuit created when two fiducial markers are engaged with a linear potentiometer.

FIGS. 4A-4D illustrate another exemplary variation of fiducial markers interacting with a linear potentiometer of a plunger of a syringe.

FIG. 5 illustrates an exemplary variation of a syringe comprising an optical sensor.

FIG. 6 illustrates another exemplary variation of a syringe comprising an optical sensor.

FIGS. 7A and 7B illustrate an exemplary variation of the occurrence of backlash when a center distance between the linear gear and the circular gear is not maintained.

FIG. 8 shows an exemplary variation of a system for determining additional movement needed to compensate for backlash via measurement of pressure.

FIGS. 9 illustrates a graph showing spikes in pressure for fluid movements of 100-200 μL to and from an expandable member.

FIG. 10 shows an exemplary variation of a mechanism to indirectly detect movement of the plunger via torque estimation.

FIGS. 11A-11C depict an exemplary variation of a movement of a circular gear and/or a linear gear based on the pressure inside the syringe pump.

FIG. 12 depicts an exemplary variation of states that a controller may execute to move the plunger of the syringe pump.

FIG. 13 is a flowchart illustrating an exemplary method of operating a syringe pump to minimize backlash.

FIG. 14 illustrates an exemplary variation of a blood flow control system.

### DETAILED DESCRIPTION

Utilizing pumps to precisely transfer fluids is critically important in many fields, and especially so in medicine. For example, many medical procedures require delivery of precise amounts of fluids, such as blood products, intravenous fluids, and medication. Additionally, in procedures utilizing devices that incorporate expandable members such as balloons, precise control of the expansion and contraction (e.g., inflation and deflation) of the expandable members may be crucial to the success of the procedure. The use of syringe pumps for precise and safe distribution of fluids may be favorable as there is already existing infrastructure for using syringes to deliver fluids.

However, manually controlling syringes can be challenging and can often result in imprecise fluid delivery, especially when dealing with small volumes of fluids. Syringes are typically manufactured in various lengths and diameters, and a wide range of doses may be delivered with relatively small movements of the plunger. Automating delivery of fluids using a syringe, or an automated syringe pump, can increase accuracy when delivering otherwise transferring fluids. However, currently available automated syringe pumps utilizing lead screws present many challenges, especially when portability (e.g., for field use) is desired.

Accordingly, described herein are new and improved systems, devices, and methods that utilize a syringe pump to transfer precise, and often small volumes, of fluids. In some variations, the syringe pumps described herein utilize a rack and a pinion that may be controlled by a motor to precisely transfer fluids to and from the syringe. While a rack and pinion-based syringe pump provides many benefits over lead-screw based devices and systems, in some instances, utilizing a rack and pinion system may necessitate addressing “backlash”. Backlash refers to the clearance between mating components and can be described as the amount of lost motion due to the clearance. (See, e.g., Niijjaawan, Niijjaawan, Modern Approach to Maintenance in Spinning, Woodhead Publishing India, 2010, Pages 272-289). Because a rack and pinion utilizes gears that are operably coupled to one another, rack and pinion-based devices are susceptible to backlash.

For example, to move the plunger of the syringe in a first translational direction, the rack and pinion may have to be rotated in a first rotational direction. To rotate the rack and pinion in the first rotational direction, the teeth of the gears may be aligned such that a first edge of a teeth of a first gear is engaged with a first edge of a teeth of a second gear. Following such an alignment, if the plunger is to be moved in a second opposite translational direction, the rack and pinion may have to be rotated in a second opposite rotational direction. To rotate the rack and pinion in the second rotational direction, the rack and pinion may have to be re-engaged. For instance, the rack and pinion may have to be re-aligned so that a second opposite edge of a tooth of the first gear is engaged with a second opposite edge of a tooth of the second gear. Therefore, the motor may have to generate additional movements to engage opposite edges of the teeth, thereby re-engaging the rack and pinion before the plunger can be moved in the second translational direction.

While some backlash is necessary to provide the clearance needed to prevent binding of gears (which can result in heat generation, noise, abnormal wear, overload, and/or failure) (See, e.g., Niijjaawan, Niijjaawan, Modern Approach to Maintenance in Spinning, Woodhead Publishing India, 2010, Pages 272-289), it may be advantageous to compensate for backlash in pumping devices and systems, such as syringe pumps, in order to increase accuracy and ensure prompt fluid delivery. For example, the flow rate of the fluid and/or the amount of fluid transferred to and from the syringe pump may, in some instances, be erroneous due to the backlash in the system. Accordingly, described herein are systems, devices, and methods that may compensate for such backlash.

Generally, the syringe pumps described herein may comprise a syringe and a pump controller. The syringe may comprise a cylindrical body, and a plunger. The plunger may comprise an actuation element (e.g., a linear gear or rack) and the pump controller may comprise an actuator (e.g., a circular gear or pinion). The actuator may be operably coupled to the actuation element, and the pump controller may also include an actuation mechanism to move the actuator, thereby moving the actuation element. For example, the pump controller may include an actuation mechanism (e.g., a motor) operably coupled to the actuator such that movement of the actuation mechanism may move the actuation element of the plunger via movement of the actuator. In some variations, the syringe pump may include a position sensor to track the position of the plunger and movement thereof during transfer of the fluid. For example, the pump controller may further comprise a position and/or motion sensor to track the position and/or movement of the motor, and thus the position and/or movement of the plunger.

Generally, the syringe pumps described herein may comprise a pump controller that may be configured to compensate for backlash in the syringe pump. For example, the controller may be configured to rotate the circular gear in a first rotational direction, thereby moving the plunger in a first linear direction, and to automatically rotate the circular gear in a second opposite rotational direction by a determined amount, where rotating the circular gear in the second rotational direction re-engages teeth of the linear gear with teeth of the circular gear and positions the plunger to be moved in a second opposite linear direction. In some variations, the pump controller may be configured to bias the circular gear in a biasing rotational direction, thereby positioning the plunger to move in a biasing translational direction, as will be described in more detail herein.

### Pump Devices

### Syringe Pumps

Syringe pumps described herein may be used to administer fluids to a patient, such as for example, precise and highly concentrated medication, precise dosages of blood products and/or intravenous fluids, etc. Bidirectional syringe pumps described herein may also be used to control a volume of an expandable member, such as a balloon, included in blood flow control devices (e.g., balloon catheters). For example, a portion of a blood flow control devices may be strategically placed within a blood vessel (e.g. aorta) of a patient. The size (e.g., volume) of an expandable member may be adjusted (e.g., inflated and/or deflated) by transferring fluid to and from a syringe in the syringe pump to partially or fully occlude the blood vessel. The amount of occlusion may regulate the blood flow to vital organs in the patient's body. This in turn may help maintain adequate oxygen delivery to the vital organs. Precisely controlling the amount of occlusion of the blood vessel allows a user to regulate the patient's blood flow and advantageously maintain adequate oxygen delivery. To precisely control occlusion, the fluid transferred to and from the expandable member may have to be precisely controlled.

In some variations, blood flow control devices may be used during procedures for removal of a thrombus from an artery or vein. Some blood flow control devices may be used to support thrombectomy catheters that are advanced into the vasculature of the brain to remove a blood clot causing a stroke. An expandable member on the end of a support or guide catheter (e.g., elongate body) may be inflated to occlude a blood vessel to momentarily stop blood flow while the thrombus or blood clot is removed. In such variations, as discussed above, the blood flow control devices may have to precisely control the delivery of fluids into and removal of fluids from the expandable member.

Described herein is a syringe pump for precise delivery of fluids. In some variations, the syringe pump may be a bidirectional syringe pump that may be used to automatically control a size (e.g., volume) of an expandable member (e.g., balloon) of a blood flow control device. More specifically, the bidirectional syringe pumps described herein more accurately control the delivery and removal of fluid from an expandable member, especially when small volumes of fluid are needed to effectuate the desired change in expandable member size. For instance, in some instances, it may be desirable to inflate or deflate in an amount as small as 1/1000^{th} of the total volume of the expandable member. For example, if expandable member can hold 25 milliliters of fluid, the bidirectional syringe pump may be used for delivering a minimum bolus of 25 microliters. The bidirectional syringe pump may be configured to transfer fluid to and from the expandable member at a flow rate of 1 milliliter per second over a pressure range from 0 to 1000 mmHg. In other variations, the bidirectional syringe pump may be used to deliver fluids (e.g., intravenous or subcutaneous fluids including medications such as vasopressor and pain medications) to a patient.

In some instances, systems and methods described herein may utilize more than one syringe pump (e.g., two, three, four, or more), one or more of which may be used to automatically control the size of an expandable member and one or more other of which may be used to deliver (e.g., automatically) fluid to a patient. In some instances, systems and methods described herein may incorporate two or more syringe pumps each configured to deliver fluid to a patient (e.g., in addition to a syringe pump used to automatically control the size of an expandable member, or in a system without a blood flow control device), each of which may deliver the same, or a different, fluid.

The syringe pumps described herein may comprise a syringe having a cylindrical body configured to contain fluid for delivery and a plunger at least partially positioned within the cylindrical body. The syringe pumps may also comprise a pump controller comprising a housing configured to removably/releasably receive the cylindrical body. The pump controller may further comprise an actuator (e.g., a circular gear) configured to engage an actuation element (e.g., a linear gear) formed from or otherwise coupled to the plunger, and an actuation mechanism (e.g., a motor) operably coupled to the actuation element to advance and/or retract the plunger, a processor, and a memory. In some variations, the pump controller may further comprise a gearbox to mechanically couple the actuation mechanism to the actuator a communication module or device, a user interface, and a power source. In some variations, the pump controller may further comprise a medication recognition interface, a sensor to detect the presence of the syringe, and/or a sensor to detect the movement and/or position of the plunger and/or motor. In variations described herein where the syringe pump is part of a blood flow control system (e.g., to deliver fluid to and remove fluid from an expandable member and/or to deliver fluids (e.g., intravenous fluids, medication, etc.) to a patient), the pump controller may be part of, or may be replaced by, a device and/or system controller that has some or all of the features and capabilities described herein with respect to the pump controller. In some variations, the system may comprise a device and/or system controller and the syringe pumps may comprise separate pump controllers.

### Cylindrical Body and Plunger

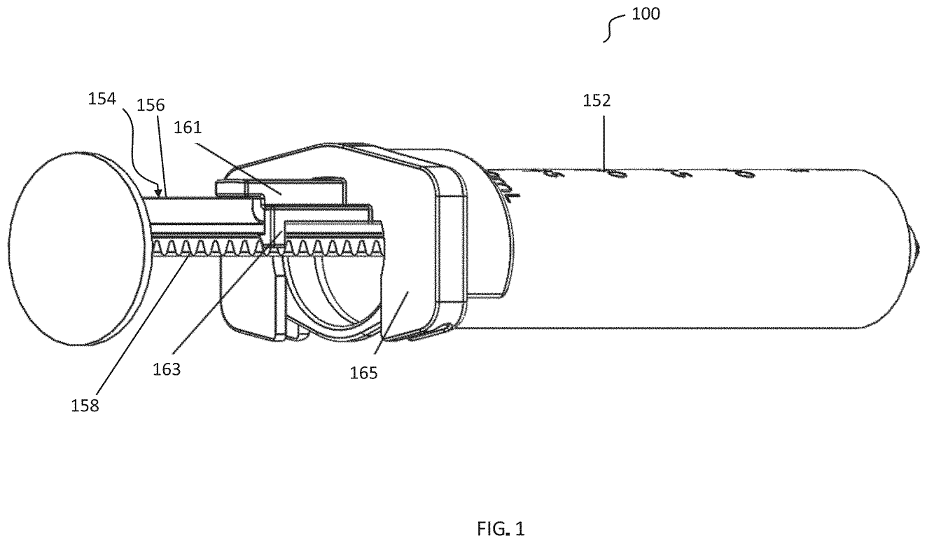

FIG. 1 illustrates an exemplary variation of a syringe 108 configured for both manual usage and for engagement with an actuator (e.g., circular gear) for automatic control. The syringe 108 may comprise a cylindrical body 152 and a plunger 154. The plunger 154 may comprise an enlarged end or head (not shown in FIG. 1) and an elongate member 156 coupled to and extending from the enlarged end. The enlarged end may be slidably positioned within the cylindrical body 152, and both the enlarged end and the elongate member 156 may be configured to move linearly within and/or relative to the cylindrical body 152. The plunger 154, and more specifically, the elongate member 156, may comprise an actuation element configured to engage with an actuator of a controller (e.g., system controller, device controller, pump controller). For example, in some variations, the elongate member 156 may comprise a linear gear 158. The linear gear 158 may be configured to engage with a circular gear (not shown in FIG. 1) partially or fully positioned within a housing of the pump controller, a housing of a system controller, and/or a housing of a device controller so as to actuate the syringe 108. In some variations, the syringe 108 may be coupled to and/or integrated with one or more sensors to identify and track a position of the plunger 154 (as described further below).

As discussed above, the syringe 108 may be automatically controlled by a controller (e.g., pump controller, system controller, and/or device controller) via the engagement of the actuator and the actuation element. For example, referring to FIG. 1 specifically, the syringe 108 may be actuated by engaging the actuator (e.g., a circular gear), with the actuation element (e.g., linear gear 158) thus forming a rack and pinion mechanism. In contrast to lead-screw drive mechanisms that may extend beyond the length of the plunger 154, the linear gear 158, and thus the rack and pinion mechanism, may be incorporated entirely within the length of the plunger 154. Accordingly, the overall footprint of the system may be reduced.

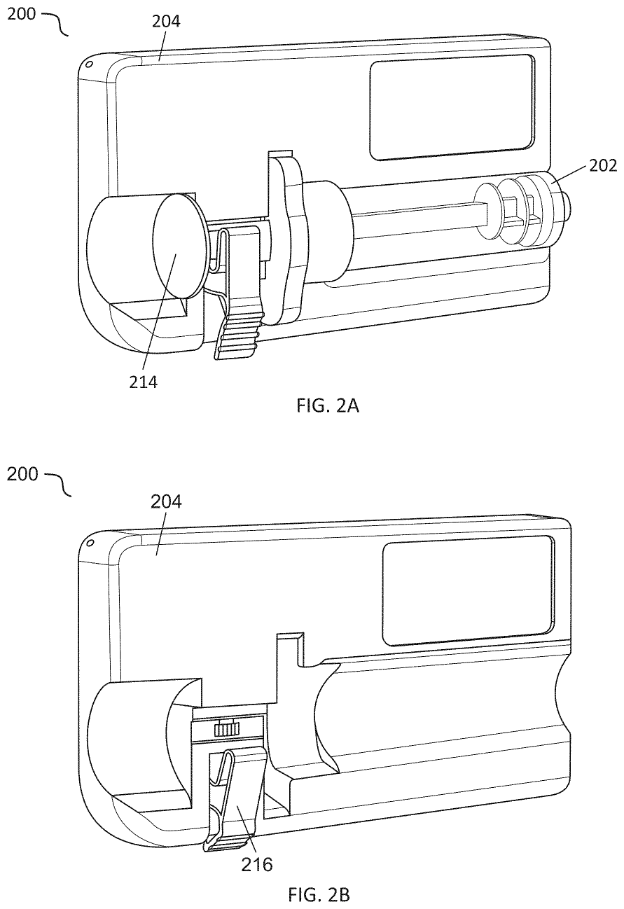

As mentioned above, a syringe pump may include a syringe, such as for example, syringe 108, and a controller (e.g., pump controller, system controller, and/or device controller) to automatically control a movement of the syringe plunger. FIGS. 2A-2C depict an exemplary variation of syringe pumps 200. The syringe pumps 200 may comprise a syringe having a cylindrical body 202 and a plunger partially disposed within the cylindrical body 202. The cylindrical body 202 may be releasably mounted into a housing 204 of a pump controller. The plunger may comprise an enlarged first end 210 positioned within the cylindrical body 202 and an elongate member (e.g., comprising linear gear 208) extending from the enlarged first end 210. The plunger may further comprise a flange or handle 214 coupled to the end of the elongate member 208. The plunger may be linearly advanceable and retractable relative to the cylindrical body 202 to deliver a fluid.

The plunger, and more specifically, the elongate member, may comprise an actuation element configured to engage with an actuator of a controller (e.g., pump controller, system controller, device controller). For example, in some variations, the elongate member itself may be configured to be a linear gear (e.g., linear gear 208) or may otherwise comprise a linear gear. For example, the linear gear may be part of or integrally formed with the elongate member. Referring again to FIGS. 2A-2C, the linear gear 208 may include a plurality of teeth 207 configured to engage a circular gear 206 so as to actuate the syringe pump. The pump controller housing 204 may contain at least partially therein the circular gear 206 and may be configured to receive the cylindrical body 202 and provide mating between the circular gear 206 and teeth 207 of the linear gear 208. For example, in some variations, the pump controller housing 204 may comprise a cavity or chamber 205 (e.g., a concave cavity formed within the housing) configured to receive the cylindrical body 202. In some variations, the cavity or chamber 205 may comprise a shape that corresponds to the shape of cylindrical body and/or plunger, such that the cylindrical body and/or plunger are easily received and held within the chamber and or cavity 205.

In some variations, one or more sensors may be employed on or used with the plunger and/or other components of the syringe pump and/or the syringe pump controller to determine a position of, and/or track movement of, one or more components and/or to detect and/or determine the amount of fluid that has been removed from or received within the cylindrical body. For example, one or more sensors may be employed to detect the position of the plunger within the cylindrical body and/or track movement of the plunger, each of which may be used to inform syringe pump movements and/or to determine the amount of fluid delivered from or received in the cylindrical body. Additionally or alternatively, one or more sensors may be employed to detect the position of and/or track movement of one or more of the motor, one or more gears in a gearbox, and the actuator (e.g., circular gear) in the controller, which may be used to inform syringe pump movements and/or to determine the amount of fluid delivered from or received in the cylindrical body. The one or more sensors may be position sensors or motion sensors, as will be described in more detail herein. In some variations, the syringe pump (e.g., the plunger) may comprise one or more sensors, the controller (e.g., pump controller, device controller, system controller) may comprise one or more sensors, or both. The sensors may be communicably coupled to a processor, such as a processor in the controller (pump controller, device controller, system controller). In some variations, the one or more sensors may include optical sensors, such as, for example, optical encoders, magnetic sensors, such as, for example, magnetic encoders, and/or contact sensors, such as, for example, linear potentiometers. In some variations, the one or more sensors may include acoustic sensor, ultrasonic sensors, etc.

As mentioned above, in some variations, the one or more sensors, such as for example, linear potentiometers, optical sensors, and the like, may be employed on or used with the plunger to detect the position of the plunger within the cylindrical body and/or track movement of the plunger. The plunger (e.g., the elongate member of the plunger) may include one or more fiducial markers (fiducials) that may be configured to interact with a sensor to detect the position of the plunger and/or track plunger movement. For example, in some variations, the plunger (e.g., elongate member of the plunger) may comprise one or more engagement pins that may be detected (e.g., via contact) by the sensor, which may be housed in the housing of the controller (e.g., device controller, pump controller and/or system controller). In some variations, the sensor (e.g., a linear potentiometer) may comprise a slider with one or more channels configured to engage with the fiducial markers. The interaction between the slider and the fiducial markers may inform the controller of the position and/or movement of the plunger and thus a volume of fluid within the cylindrical body of the syringe pump.

The fiducial markers may be any suitable marker configured to be detected (e.g., via contact, optically, magnetically) by a sensor. For instance, the fiducial marker may be an engagement pin of any suitable shape (e.g., triangular, circular, cylindrical, elliptical, etc. cross-sectional shape). In some variations, the plunger may include a single fiducial marker to detect the position of the plunger and/or to track plunger movement. In some such variations, the sensor (e.g., slider of the linear potentiometer) may be configured to contact the entire length of the plunger. In other words, the linear potentiometer (e.g., the slider with one or more channels) may be configured to span the entire length of the plunger such that the single fiducial marker contacts at least a portion of the linear potentiometer at all times. A change in voltage and/or a change in resistance caused due to the single fiducial marker contacting the linear potentiometer may be indicative of the position of the plunger and thus volume of fluid in the syringe pump. In some variations, the plunger may comprise a plurality of fiducial markers (e.g., two, three, four, five, six, seven, eight, nine, ten or more) and/or a plurality of pairs or sets of fiducial markers (e.g., two, three, four, five, six, seven, eight, nine, ten or more). In some variations, the fiducial markers may be positioned along a single line, while in other variations the fiducial markers may be positioned along two or more lines. For example, in some variations, the fiducial markers may be spaced asymmetrically along two or more lines. In some variations, the distance between two fiducial markers on the plunger may be unique (e.g., the distances between each fiducial marker and each adjacent fiducial marker may be different).

In some variations, the fiducial markers may be offset from one another and/or may be spaced asymmetrically along the elongate member, such that when the fiducial markers contact/engage with the sensor (e.g., a linear potentiometer), they may provide a unique indicator or “code” for every position along the elongate member of the plunger. In some variations, the fiducial markers may additionally or alternatively assist the controller in determining an initial volume of the syringe. For example, the syringe may be inserted into or positioned within the controller (e.g., device controller, pump controller and/or system controller) housing at any volume suitable for a particular procedure. Since the volume may be different at different times and/or for different subjects, the controller may determine the initial volume of the syringe based on the interaction between the fiducial marker(s) and the sensor (e.g., interaction between the fiducial marker(s) and slider of the linear potentiometer). The interaction may inform a system (e.g., a blood flow control system) comprising the syringe of the total volume of fluid within the cylindrical body of the syringe upon coupling to (e.g., insertion or placement in) the controller housing.

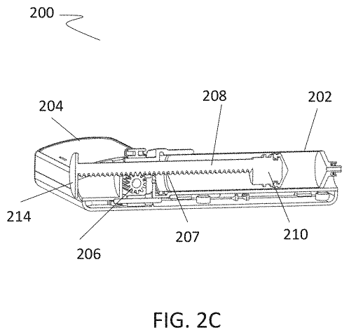

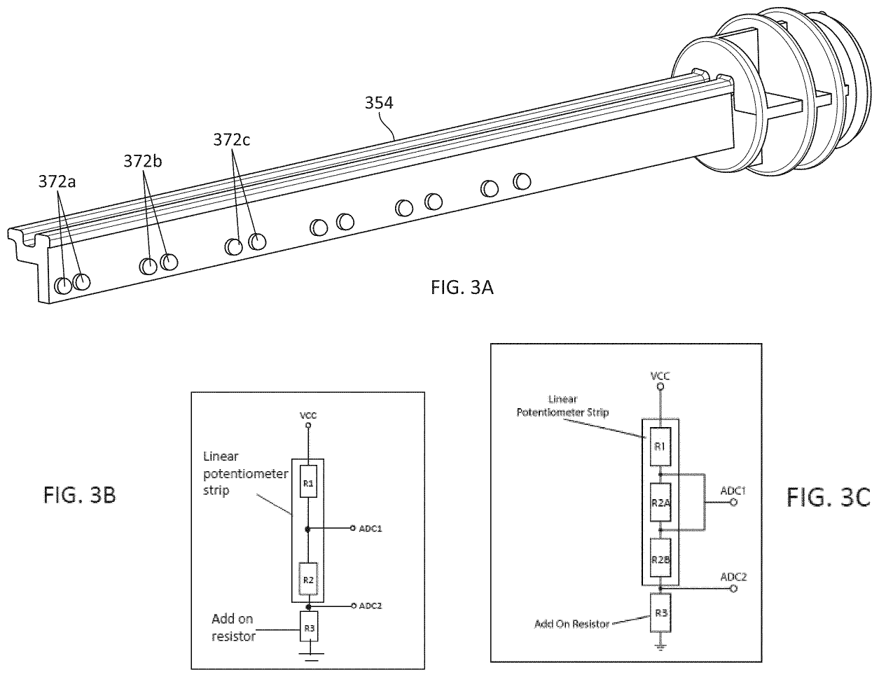

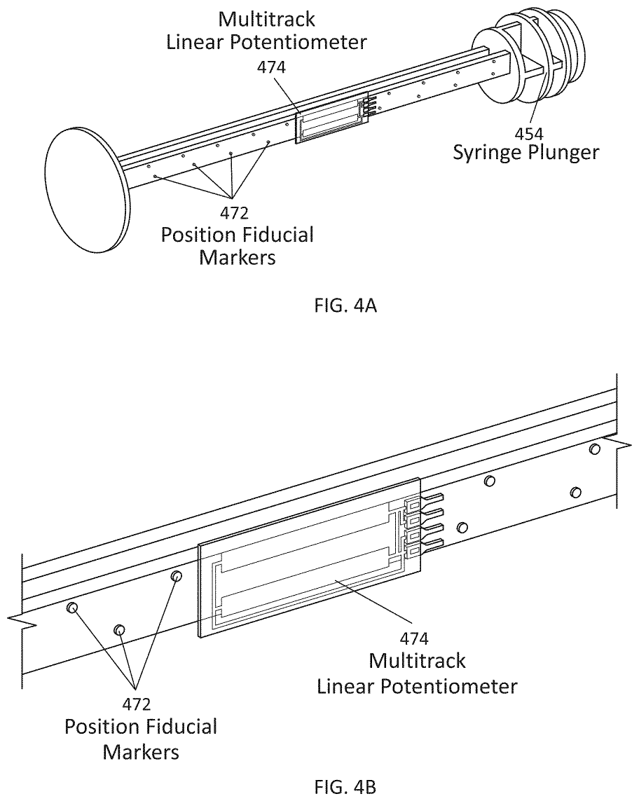

FIG. 3A illustrates an exemplary variation of a plunger of a syringe included in a syringe pump comprising fiducial markers. As shown there, the elongate member of the plunger 354 may comprise a plurality of fiducial markers 372a, 372b, 372c (collectively referred to as fiducial markers 372) in the form of cylindrical pins. The plurality of fiducial markers 372 may have any suitable cross-sectional shape, such as, for example, circular, triangular, elliptical, square, a combination thereof, and the like, and any height suitable to contact or otherwise interact with a sensor. The fiducial markers 372 may be positioned along the length of the elongate member and may be grouped in pairs, although need not be. In some variations, the fiducial markers 372 may be non-linearly spaced. For instance, the spacing between each fiducial marker may be unique. Additionally or alternatively, the spacing between two fiducial markers in each pair of fiducial markers may be unique. For example, the distance between the two fiducial markers of the pair 372a may be different from the distance between the two fiducial markers of the pair 372b. In some variations, the distance between each pair of fiducial markers may be unique. For example, in FIG. 3A, the distance between the pair 372a and the pair 372b may be different from the distance between the pair 372b and the pair 372c. In some variations, the spacing between the fiducial markers may ensure that no more than two markers may engage with the linear potentiometer at a time. In some variations, the fiducial markers may be positioned on a single track (i.e., along a line) along the length of the elongate member of the plunger, and/or along a plurality of tracks (e.g., two, three, or more).

FIG. 3B illustrates an exemplary variation of an electrical circuit created when one fiducial marker is engaged with a linear potentiometer. FIG. 3C illustrates an exemplary variation of an electrical circuit created when two fiducial markers are engaged with a linear potentiometer. In some instances, it may be advantageous to utilize linear potentiometers to monitor the position and/or movement of the plunger because linear potentiometers provide precise measurements and generally have low power consumption. As depicted in FIG. 3B and in FIG. 3C, a linear potentiometer may generally comprise a slider with one or more channels. To create an electrical circuit, the linear potentiometer may also comprise a resistive element (e.g., resistive strip, resistor, etc.) with two or more terminals. The terminals of the resistive element may be connected such that when the slider of the linear potentiometer engages with fiducial marker(s), a voltage divider may be created.

For example, a total resistance of the linear potentiometer may be a fixed value. In FIG. 3B and FIG. 3C, the linear potentiometer may have three terminals. A resistance between a first terminal and a second terminal of the linear potentiometer is depicted as R1, and the resistance between the second terminal and a third terminal of the linear potentiometer is depicted as R2. When a single fiducial marker engages with the slider of the linear potentiometer, the total resistance of the linear potentiometer may be split into two resistances, such as for example, R1 and R2. Accordingly, a position of the fiducial marker that engages with the linear potentiometer in FIG. 3B may be determined from a ratio between the resistance R1 and R2. That is, the ratio between R1 and R2 in FIG. 3B may correlate to a position of the fiducial marker that engages with the linear potentiometer.

FIG. 3C illustrates an exemplary variation of an electrical circuit created when two fiducial markers are engaged with a linear potentiometer. In FIG. 3C, the second fiducial marker of the two fiducial markers may split the resistance R2 into R2A and R2B. The second fiducial marker may create a short around R2A. Therefore, the resistance between the first terminal and the second terminal may still be R1. The resistance between the second terminal and the third terminal may be R2B and the resistance between the first terminal and the third terminal may be the sum of R1 and R2B.

For both FIG. 3B and FIG. 3C, an additional resistor R3 may connected between the third terminal of the linear potentiometer and the circuit's ground. Accordingly, when a voltage is applied to the first terminal VCC, the voltage at the second terminal may be measured by ADC1 and the voltage at the third terminal may be measured by ADC2. If one fiducial marker is engaged with the linear potentiometer, such as in FIG. 3B, then the voltage measured by ADC2 may be a constant value based on the sum of resistances R1 and R2 (the sum may be constant since the total resistance of the potentiometer is constant and is split into R1 and R2) and resistance R3. The voltage measured at ADC1 may vary based on the position of the fiducial marker. Therefore, voltage ADC1 may be indicative of the position of the fiducial marker. In some variations, the voltage ADC1 may be transmitted to a controller (e.g., system controller, device controller, and/or pump controller). The position of the plunger determined from ADC1 may be used to calculate a position of the plunger and/or may be used to track a movement of the plunger.

However, if two fiducial markers are making contact with the linear potentiometer, such as in FIG. 3C, then R2 may decrease because of the shorting around R2A. The voltage measured at ADC2 may be correlated to the distance between the two fiducial markers while the voltage measured at ADC1 may be correlated to the position of a first fiducial marker of the two fiducial markers that may be engaged with the linear potentiometer. Therefore, an absolute position of the plunger may be derived from voltages measured at ADC1 and ADC2.

For instance, the spacing of the fiducial markers may be key to determining specifically which pair of fiducial makers may be currently engaged with the linear potentiometer. In addition, because the entire span and spacing between fiducial markers is known, it may be possible to determine the position of the plunger when even a single fiducial marker engages with the potentiometer. For example, since the entire span and the distances between the fiducial markers are known, a look up table may be generated to associate the voltage/resistance when each of the fiducial marker engages with the potentiometer, and the look up table may be used to determine the position (e.g., absolute position) of the plunger.

Since the variation in FIG. 3A comprises pairs of fiducial markers spaced across the length of the plunger, the length of the linear potentiometer may be an order of magnitude shorter than the length of the plunger. This is because when a plunger comprises only a single fiducial marker, the potentiometer (e.g., the slider of the potentiometer) may need to span the length of the plunger so that for every movement of the plunger at least some portion of the potentiometer (e.g., slider of the potentiometer) engages with the single fiducial marker. However, since in FIG. 3A, pairs of fiducial markers are spaced across the length of the plunger, even with a potentiometer of a shorter length, the potentiometer may engage with one or more fiducial markers for every movement of the plunger. The result of this design is that the overall form factor may be decreased, and the resolution of movement sensing may be substantially increased.

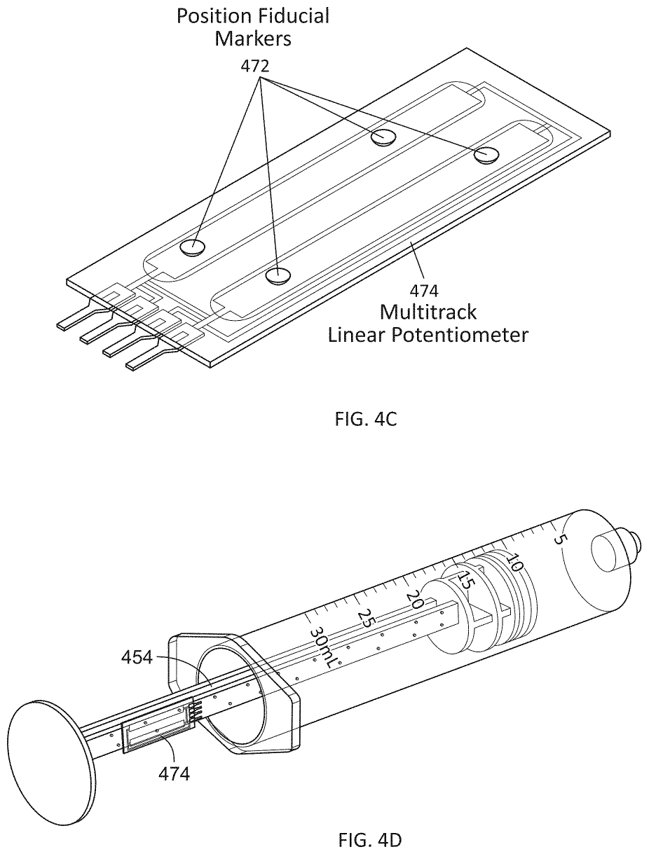

FIGS. 4A-4D illustrate another exemplary variation of fiducial markers interacting with a linear potentiometer. In this variation, the fiducial markers may be spaced asymmetrically along multiple tracks instead of a single track (e.g., single line). As discussed above, in some variations, the fiducial markers may be offset from one another and/or may be spaced asymmetrically along the elongate member, such that when the fiducial markers contact/engage with the sensor (e.g., linear potentiometer), they may provide a unique indicator or “code” for every position along the elongate member of the plunger.

In some variations, the sensor may be a multitrack linear potentiometer. That is, the sensor may comprise multiple channels to engage with the fiducial markers. More specifically, the sensor may be a linear potentiometer comprising a slider with two or more channels. Each channel may be configured to engage with one or more fiducial markers. For example, if the fiducial markers are placed on two tracks along the length of the plunger, then a first channel of the linear potentiometer may engage with fiducial markers on a first track and a second channel of the linear potentiometer may engage with fiducial markers on a second track

FIG. 4A and 4B depict a linear potentiometer 474 engaging with engagement pins 472 positioned on multiple tracks (e.g., two). As seen in FIG. 4B, the linear potentiometer 474 comprises a slider with two channels (e.g., a multitrack potentiometer). When a multitrack linear potentiometer 474 is provided in or on the controller (e.g., system controller, device controller, and/or pump controller) housing to engage or otherwise interact with fiducial markers on the elongate member of the plunger 454, as shown in FIGS. 4A and 4B, engagement pins (fiducial markers) 472 on the side of the plunger 454 may contact the multitrack linear potentiometer 474 and provide precise measurements of actual syringe movement. Referring to FIGS. 4C and 4D, a series of offset engagement pins (fiducial markers) spaced asymmetrically along the length of the elongate member of the plunger may connect with the channels on the linear potentiometer to thereby provide a “code” for every position along the syringe. More specifically, the engagement pins 472 may be spaced in multiple lines along the length of elongate member. When a multi-channel potentiometer engages with one or more fiducial markers, the unique spacing between the fiducial markers may cause the linear potentiometer to generate a unique code indicative of the position of the plunger. In this manner, the movement of the plunger and/or the position of the plunger may be determined.

In some variations, the resistive element of the linear potentiometer described above may be a part of a resonator circuit that changes frequency in proportion to the change in the resistance. A controller (e.g., system controller, device controller, and/or pump controller) may measure the frequency of the resonator circuit to determine a position and/or movement of the plunger. In some variations, instead of the resistive element, the linear potentiometer may include a capacitive element that may change capacitance based on the engagement of the linear potentiometer with the fiducial markers. In such variations, the change in capacitance may change the frequency of the resonator circuit. The controller may measure the frequency of the resonator circuit to determine a position and/or movement of the plunger. In some variations, instead of the resistive element, the linear potentiometer may include one or more indicators that may change inductance based on the engagement of the linear potentiometer with the fiducial markers. In such variations, the change in inductance may change the frequency of the resonator circuit. The controller may measure the frequency of the resonator circuit to determine a position and/or movement of the plunger. In some variations, in addition to the fiducial markers above, a second set of fiducial markers and a second linear potentiometer may be positioned such that the second linear potentiometer and the second set of fiducial markers may provide an indication as to the position of the plunger relative to the cylindrical body.

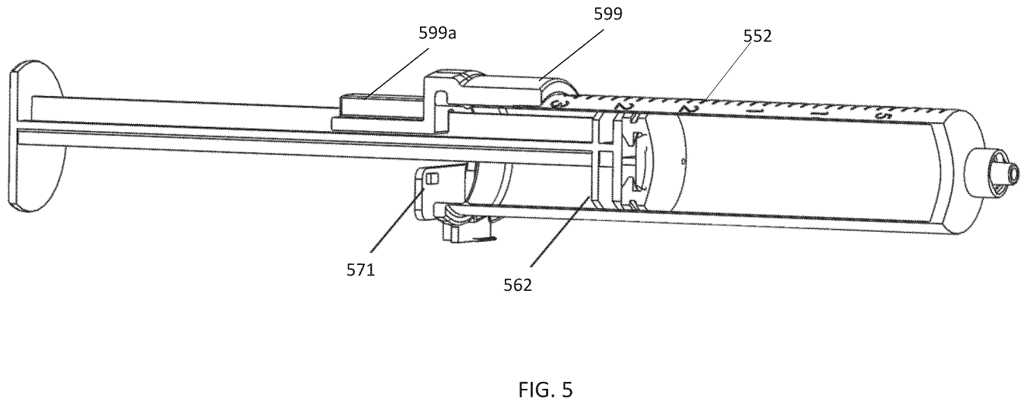

In some variations, as mentioned above, optical sensors may be used to detect the position of the plunger within the cylindrical body and/or to track movement of the plunger. FIG. 5 illustrates an exemplary variation of an optical sensor 571 configured to detect the position of the plunger and/or to track the movement of the plunger. In some variations, the optical sensor 571 may be coupled directly to the pump as shown in FIG. 5. For instance, the optical sensor 571 may be coupled to and/or integrated with a proximal end of the cylindrical body 552. In some variations, the syringe pump may further comprise a sleeve 599 coupled to or formally integrally with the proximal end of the cylindrical body, and the sensor may be attached to the sleeve. For example, in some variations, the sleeve 599 (e.g., a distal portion 599b) may be configured to receive the proximal end of the cylindrical body and may be coupled to the proximal end of the cylindrical body in any suitable manner, such as, for example via snap-fit connectors or other mechanical connectors (e.g., screws, pins, etc.), an interference fit, adhesive, plastic welding, and/or the like. In some variations, a portion (e.g., a proximal portion 599a) of the sleeve 599 may be configured to contact and/or support a portion of the plunger. The optical sensor 571 may be positioned on the sleeve 599 such that the optical sensor 571 may face towards the cylindrical body and towards the enlarged end 562 of the plunger. As the plunger and consequently the enlarged end 562 translates within the cylindrical body, light reflected from the enlarged end 562 may be received by the optical sensor 571. The volume of the fluid in the pump may be determined based on the detected reflected light. For instance, the sensor 571 may transmit an amount of reflected light to a controller (e.g., system controller, device controller, and/or pump controller) that may determine a position of the enlarged end 562 based on the amount of reflected light. The controller may use the position of the enlarged end 562 to determine a volume of fluid in the pump. The volume of fluid in the pump may be used to determine the amount of fluid received in or delivered from the cylindrical body and/or to track the position and/or movement of the plunger.

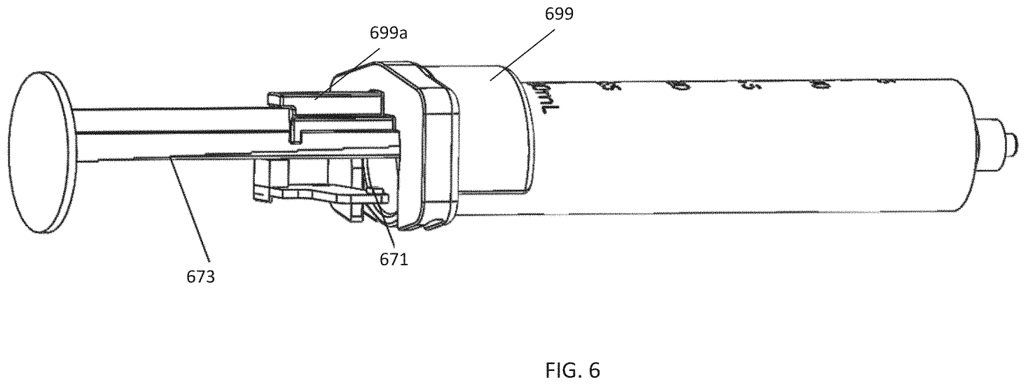

FIG. 6 illustrates another exemplary variation of an optical sensor 671 configured to detect the position of the plunger and/or to track the movement of the plunger. In FIG. 6, the optical sensor 671 may be positioned on a sleeve 699 such that the optical sensor 671 may be oriented to face towards the plunger. A proximal end of the cylindrical body may be configured to receive the sleeve 699. Additionally or alternatively, the sleeve may be attached to the proximal end of the cylindrical body in any manner described with respect to FIG. 5. As discussed, the optical sensor 671 may be positioned on the sleeve 699 such that the optical sensor faces the plunger. For instance, the optical sensor 671 may face a bottom surface of the plunger. The plunger may include a marker 673 configured to reflect light in a manner that allows the optical sensor 671 to determine the position of the plunger based on the reflected light. In some variations, the marker 673 may be a step-shaped marker spanning the length of the plunger. For instance, the step-shaped marker may include inclined surfaces spanning the length of the plunger such that light may reflect off the marker differently based on the position of the marker relative to the optical sensor 671. The optical sensor 671 may detect the light reflected from different portions of the step-shaped marker 673 and these measurements may be used (e.g., via a controller described herein) to determine a position of and/or track movement of the plunger based on the detected light.

In some variations, magnetic sensors may be used to detect the movement and/or position of the plunger. The magnetic sensor may comprise a magnetic reader that may detect changes in magnetic fluxes. The magnetic sensor may be attached and/or coupled to the plunger. For instance, one or more magnets may be mounted on the plunger at non-uniform distances and with non-uniform polarities. As a non-limiting example, a first magnet may be mounted at a first distance from an end of the plunger, a second magnet may be mounted at a second distance from the end of the plunger, and a third magnet may be mounted at a third distance from the end of the plunger. The polarity of at least one of the three magnets may be different, such as for example, if the first magnet has a north polarity, the second magnet has a south polarity and the third magnet has either a north or a south polarity. When the plunger moves, the magnetic reader may detect a change in the magnetic flux. This change may be processed by a controller (e.g., system controller, device controller, and/or pump controller) to determine a position and/or movement of the plunger. For example, the change in magnetic flux may be detected and processed by an integrated circuit that may generate a voltage signal that may be processed by the controller. The voltage signal may generate pulses (referred to as encoder steps) to indicate the movement of the plunger.

In some variations, a sensor configured to detect the movement and/or position of the plunger may incorporate a roller or secondary gear. The roller or secondary gear may be configured to contact the plunger. This roller or secondary gear may have a higher ratio than the circular (pinion) gear, so a very small change in the position of the circular gear will result in a large rotation of the roller or secondary gear. The rotation of the roller or secondary gear may be detected for example, using magnetic, optical, and/or capacitive sensors. The amount of rotation of the roller or secondary gear may be processed by a controller (e.g., system controller, device controller, and/or pump controller) to determine a position and/or movement of the plunger.

In some instances, the syringe pumps described herein may include one or more sensors configured to detect a position and/or movement of a component other than the plunger and may utilize that position and/or movement as a corollary for the position and/or movement of the plunger, or may otherwise utilize the position and/or movement of that component to inform a determination of the position and/or movement of the plunger, an amount of fluid transferred (delivered or received), and/or size of an expandable member. For example, in some variations, the syringe pumps described herein may comprise a sensor configured to detect a position and/or movement of the actuation mechanism, such as, for example, the motor of the syringe pump. For example, the syringe pumps described herein may include optical sensors such as optical encoders and/or magnetic sensors such as magnetic encoders configured to detect a position and/or movement of the motor, which in turn may be used to determine movement and/or position of the plunger, as will be discussed in more detail herein with respect to the pump controller. In some variations, the sensors described herein may be configured to detect a position of and/or track a movement of one or more other components of the syringe pump (e.g., gearbox, gears in the gearbox, an actuation element (e.g., linear gear), an actuator (e.g., circular gear), etc.).

### Pump Controller

The syringe pump may comprise a pump controller configured to control automated actuation of the pump and delivery of fluid therefrom (or reception therein). The pump controller may further comprise an actuator (e.g., a circular gear) configured to engage an actuation element (e.g., linear gear), an actuation mechanism (e.g., a motor) operably coupled to the actuation element (e.g., the linear gear) to advance and/or retract the plunger, a gearbox to mechanically couple the actuation mechanism to the actuator (e.g., the circular gear), a processor (e.g., microprocessor), a memory, a communication module or device, a user interface, and a power source. In some variations, the pump controller may further comprise a medication recognition interface, a sensor to detect the presence of the syringe, a sensor to detect the movement and/or position of the plunger or other component of the syringe pump (e.g., actuation mechanism, actuator), and a lock.

The pump controller may comprise a housing configured to receive the cylindrical body and plunger therein. For example, in some variations, the housing may comprise a cavity, chamber (open or closed) or other opening configured to receive the cylindrical body and plunger and to hold the cylindrical body in place relative to the controller housing. The pump controller may further comprise a lock (e.g., a latch) coupled to the pump controller housing. and the lock may be configured to releasably couple the cylindrical body and/or the plunger (e.g., the elongate member of the plunger) to the housing to maintain the position of the cylindrical body relative to the controller housing. The lock may also assist in aligning the actuator (e.g., the circular gear) and the actuating element (e.g., the linear gear).

The controller housing may at least partially house an actuator, which may comprise a circular gear (pinion gear), configured to engage an actuation element of the plunger, and the actuation mechanism operably coupled to the actuator, such as, for example, via a gearbox. As described in more detail herein, the actuation element may comprise a linear gear (rack) and may be coupled to or integral with the plunger. The actuator may be configured to engage with the actuation element, such that rotational movement of the actuator may be translated to linear movement via the actuation element. In this manner, rotation of the actuator may linearly advance the plunger. The processor/memory of the pump controller may be configured to control actuation of the actuator (e.g., circular gear) based on the position and/or movement of one or more components of the syringe pump, such as, for example, the position and/or movement of the actuation mechanism, the actuator, and/or the plunger detected by one or more sensors.

The pump controller may comprise one or more processors. Generally, the one or more processors (e.g., CPU) may process data and/or other signals to control one or more components of the pump. The processor may be configured to receive, process, compile, compute, store, access, read, write, and/or transmit data and/or other signals. In some variations, the processor may be configured to access or receive data and/or other signals from one or more of a sensor and a storage medium (e.g., memory, flash drive, memory card). In some variations, the processor may be any suitable processing device configured to run and/or execute a set of instructions or code and may include one or more data processors, image processors, graphics processing units (GPU), physics processing units, digital signal processors (DSP), analog signal processors, mixed-signal processors, machine learning processors, deep learning processors, finite state machines (FSM), compression processors (e.g., data compression to reduce data rate and/or memory requirements), encryption processors (e.g., for secure wireless data and/or power transfer), and/or central processing units (CPU). The processor may be, for example, a general-purpose processor, Field Programmable Gate Array (FPGA), an Application Specific Integrated Circuit (ASIC), a processor board, and/or the like. The processor may be configured to run and/or execute application processes and/or other modules, processes and/or functions associated with the system. The underlying device technologies may be provided in a variety of component types (e.g., metal-oxide semiconductor field-effect transistor (MOSFET) technologies like complementary metal-oxide semiconductor (CMOS), bipolar technologies like generative adversarial network (GAN), polymer technologies (e.g., silicon-conjugated polymer and metal-conjugated polymer-metal structures), mixed analog and digital, and/or the like.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

Generally, the pump controllers described herein may comprise a memory configured to store data and/or information. In some variations, the memory may comprise one or more of a random access memory (RAM), static RAM (SRAM), dynamic RAM (DRAM), a memory buffer, an erasable programmable read-only memory (EPROM), an electrically erasable read-only memory (EEPROM), a read-only memory (ROM), flash memory, volatile memory, non-volatile memory, combinations thereof, and the like. In some variations, the memory may store instructions to cause the processor to execute modules, processes, and/or functions associated with an external device, such as, for example, a blood flow control device, such as signal waveform generation, expandable member control, data and/or signal transmission, data and/or signal reception, and/or communication. Some variations described herein may relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes.

The pump controller may further comprise an actuator (e.g., circular gear) and an actuation mechanism such as a motor, each of which may be at least partially positioned within the pump controller housing. The motor may be operably coupled to the actuator (e.g., the circular gear). In some variations, the motor may be directly coupled to the actuator, while in other variations, the pump controller may further comprise a gearbox containing one or more gears, through which the motor may be coupled to the actuator. Put differently, in some variations, the motor may be operably coupled to the circular gear via the gearbox. In variations comprising a motor, the motor may be any suitable motor, such as, for example, a stepper motor. The gearbox may be contained within the controller housing or mounted on the controller housing via, for example, mechanical connectors such as screws.

In some variations, it may be advantageous to select the actuator (e.g., the circular gear) and in variations comprising a gearbox, the gearbox, to achieve a high gear ratio and fine pitch as this may provide for precise control of the pump, including control more precise than is possible manually. More specifically, the gear ratio of the gearbox may be selected to allow for the high gear ratio of the actuator. In some variations, the gear ratio of the gearbox may be between about 40:1 and about 150:1. For example, the gear ratio may be between about 130:1 and about 60:1, between about 115:1 and about 75:1, between about 105:1 and about 85:1, between about 97:1 and about 82:1, between about 95:1 and about 85:1, between about 92:1 and about 87:1 (including all values and sub-ranges therein). In some variations, the gear ratio of the gearbox may be about 90:1, such as for example, about 90.75 to 1, about 90.50 to 1, or about 90.25 to 1.

In some variations, the ratio of the rotation of the actuator (e.g., the circular gear) to the linear translation of the actuation element (e.g., the linear gear) may depend on the pitch diameter of the actuator (e.g., the circular gear). In variations comprising a circular gear, the pitch diameter of the circular gear may be between about 4 mm and about 20 mm, between about 6 mm and about 18 mm, between about 8 mm and about 16 mm, between about 10 mm and about 15 mm (including all values and sub-ranges therein). In some variations, the pitch diameter of the circular gear may be about 14.4 mm. In some variations the module (metric pitch) of the circular gear may be between about 0.4 mm and about 1.0 mm, between about 0.5 mm and about 0.9 mm, between about 0.6 mm and about 0.8 mm, or between about 0.7 mm and about 0.75 mm (including all values and sub-ranges therein). In some variations, the module may be about 0.4 mm.

The pump controller may also include a wired or wireless communication module or device configured to communicate with another device, e.g., a tablet, a smart phone, etc., that may control the pump. In some variations, the communication module or device may be configured to communicate with external devices, such as, for example other treatment devices like a blood flow control device, and/or the communication module or device may be configured to communicate with a main network or hub via which the pump (and optionally other treatment devices) may be controlled. The communication module may be configured to communicate via wireless protocols such as Bluetooth, Wi-Fi, and/or a cellular network and/or via wired protocols such as UART, RS232, Ethernet, etc. The communication interface includes the ability to connect to other devices for conveying system state and reporting of data, as well as getting input for changing state or configuration.

The pump controller may also comprise a user interface communicably coupled to the processor and/or memory such that the processor may receive data related to the pump (e.g., position of the plunger, displacement of the plunger, volume of fluid inside the cylindrical body, volume of fluid delivered, volume of fluid received, a combination thereof, and/or the like) and the user interface may display data. The user interface may include a display, such as an LCD or OLED panel, one or more LEDs, and/or an audio output, such as an audio speaker. It may also include haptic output. The user interface may also be configured to receive user input, such as from physical buttons and/or a touchscreen of the user interface. The user interface (e.g., buttons or touchscreen of the user interface) may be used to provide information to the pump, such as, for example, a desired volume of fluid to be delivered and/or a type of fluid to be delivered (e.g., a type of medication).

The pump controller (e.g., the processor of the pump controller) may instruct or otherwise activate the motor to deliver fluid from the pump and/or, in some variations, draw fluid into the pump. For example, the processor may be configured to activate an actuation mechanism, such as a motor, for a desired duty cycle and period. For example, in variations comprising a stepper motor, the processor may be configured to activate the motor to perform a specified number of steps every certain period of time (e.g., 1 step every 10 milliseconds), and/or to perform a specified number of steps during a certain period of time (e.g., 10 steps during the course of 10 milliseconds), and/or to delay a certain period of time before performing a specified number of steps (e.g., wait 500 milliseconds before preforming another 10 steps).

The pump controller may further comprise a power source. The power source may be housed within the pump controller housing. The power source may comprise an integrated battery pack and/or a connection to an external battery pack. In some variations, the pump controller may further comprise an AC-to-DC converter. In some variations, the pump controller may be electrically coupled to an external power source. In variations comprising a battery pack, the batteries may or may not be rechargeable.

As discussed above, the pump controller may include a sensor configured to detect a presence of the syringe pump. In some variations, the sensor may comprise a syringe recognition circuit. In some variations, in addition to detecting the presence of the syringe pump, the syringe recognition circuit may also be configured to detect the type of syringe pump that is being used and/or identify details on medication that may be contained within (e.g., prefilled in) the cylindrical body. In variations comprising a syringe recognition circuit, the pump controller may comprise a mechanical switch that may be electrically coupled to the processor of the pump controller. When the syringe pump is inserted into the cavity, chamber (open or closed) or otherwise coupled to the controller housing, the mechanical switch may close an electrical path. The processor may therefore detect that the syringe pump has been properly inserted into the cavity, chamber (open or closed) or otherwise coupled to the controller housing. The processor may also detect the type of syringe pump and the amount of medication based on an output voltage from the syringe recognition circuit. In some variations, the recognition circuit may include optical recognition of a barcode or QR code, or an electronic circuit to read a memory chip positioned to the side of the syringe.

As discussed above, the pump devices described herein may include sensors configured to detect the movement and/or position of the plunger. The plungers used in the syringe pumps described herein may be of various sizes, depending on the application, and the sensors may be configured to detect the movement and/or position of the plunger across the various potential sizes. The sensors may be configured to provide confirmation on whether the plunger has moved an intended amount. For example, as discussed above, the syringe pumps may include optical sensors such as optical encoders, magnetic sensors such as magnetic encoders, contact sensors such as linear potentiometer, etc. to detect movement and/or position of a plunger.

In some variations, the syringe pumps described herein may include one or more sensors configured to detect a position and/or movement of a component other than the plunger. For example, the syringe pumps described herein may include optical sensors such as optical encoders and/or magnetic sensors, such as magnetic encoders to detect the position and/or movement of an actuation mechanism such as a motor, which in turn may, in some variations, be used to determine movement and/or position of the plunger. In other variations, the syringe pumps described herein may utilize the position and/or movement of the motor to control or otherwise inform fluid transport without determining a particular position and/or movement of the plunger associated with that motor's position and/or movement respectively. Sensors configured to detect the position and/or movement of the motor may be included in the controller (pump controller, system controller, device controller). For example, these sensors may be coupled (e.g., mechanically coupled and/or optically coupled, or magnetically coupled) to the motor. The sensors may detect a change in reflected or transmitted light (e.g., optical sensors) or a change in magnetic fluxes (e.g., magnetic sensors) due to movement of the motor. This change may be analyzed by the processor of the controller (e.g., pump controller, device controller, system controller). The processor may determine an amount of motor movement (e.g., for a stepper motor, a number of steps the motor took) based on these changes. In some variations, the sensors may detect an amount of movement every certain period of time (e.g., a number of steps taken by the motor every certain period of time). For example, the sensors may detect an amount of movement every 10 seconds, every 9 seconds, every 8 seconds, every 7 seconds, every 6 seconds, every 5 seconds, every 4 seconds, every 3 seconds, every 2 seconds, every second, every 0.5 seconds, every 0.1 seconds, every 0.05 seconds, every 0.01 seconds, every 0.005 seconds, every 0.001 seconds, every 0.0005 seconds, or 0.0001 seconds. Additionally or alternatively, such sensors may have high resolution relative to the motor. That is, such sensors may be configured to indicate a higher number of steps for every physical movement of the motor. As discussed above, the sensors may generate pulses and/or encoder steps that may be indicative of the movement of the motor. As a non-limiting example, encoder steps may be generated such that one physical step of the motor may be indicated by 20 encoder steps. For instance, the sensors may be configured such that the processor may indicate 20 steps for every physical movement of the motor. The pump controller may also be operably coupled to a medication recognition device or interface that may be configured to provide information to the processor about the fluid (e.g., medication) within the cylindrical body. This information may include the type of fluid (e.g., medication), the concentration of the fluid (e.g., medication), the maximum flow rate for the fluid (e.g., medication). In some variations, this information may also include the expiration data for the medication, and/or authentication codes for the medication. Some or all of these parameters may be presented to the user via the user interface. If the user attempts to set a flow rate for the medication delivery that is greater than the maximum flow rate, the processor may notify the user, refuse that setting, and/or require a secondary user authentication. Similarly, if the medication is beyond the expiration date or the authentication codes don't show it to be from a valid manufacturing lot, the processor may notify the user, refuse that setting, and/or require a secondary user authentication.

The syringe pump may further comprise various safety mechanisms. For example, the syringe pump may include one or more of: encryption and authentication on communication between the syringe pump and other devices and/or a network (e.g., any remote data logging and control services), redundant processors and/or redundant algorithms, redundant sensors to detect the presence of the syringe, and/or the position and/or movement of the plunger or other syringe pump components such as the motor or actuator, detection of a state of the syringe pump (e.g., cylindrical body empty, full, partially full), and detection of a fault in the syringe pump (e.g., pump controller), such as, for example, a fault in the motor.

### Backlash

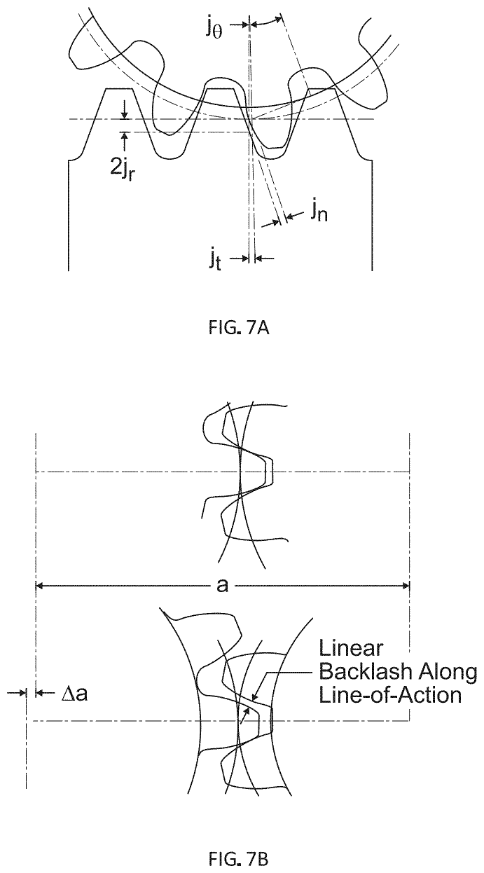

FIGS. 7A and 7B illustrate an exemplary variation of the occurrence of backlash. As described above, backlash may occur when there exists a clearance or gap between mating teeth of the gears. For instance, in FIG. 7A, the clearance between the teeth surfaces and/or edges is depicted as 2jr. In FIG. 7B, the clearance between the teeth surfaces and/or edges is depicted as Δa. Such a clearance may cause backlash. Put differently, if there is a clearance between the gear teeth, then backlash may occur.

As discussed above, the syringe pump may include one or more gears to control fluid delivery to and from a syringe. The syringe pump may include a syringe that may be operably coupled to a pump controller comprising an actuator that engages with an actuation element of the syringe to actuate the syringe. In some variations, as discussed in detail herein, the actuator may comprise a circular gear and the actuation element may comprise a linear gear. The circular gear may be operatively coupled to a motor that in turn may be coupled to a processor of a controller (e.g., pump controller, device controller, system controller). In some variations, a gearbox may also be included between the motor and the circular gear. For instance, the gearbox may couple the motor to the circular gear. In these variations, the gearbox may change the ratio between the motor's rotation and the circular gear's rotation. In some variations, the gearbox may also reduce the torque required by the motor (e.g., when the pressure inside the syringe is high). In some variations, the gearbox may reduce the torque required by the motor to move the plunger. For example, when the pressure inside the syringe is high, the pressure may push against the plunger. A gearbox with high gear ratio may reduce the torque that may be required to move the plunger against this pressure.

The linear gear and the circular gear of the syringe pump may each include a set of teeth. One or more teeth of the linear gear may mate with one or more teeth of the circular gear. For example, at least a portion of an edge of one or more teeth of the linear gear may contact at least a portion of an edge of one or more teeth of the circular gear. Although mated teeth may contact each other, they may not necessarily engage the linear gear with the circular gear. That is, a rotational movement of the circular gear may not result in a translational movement of the linear gear when they are not engaged. To engage the linear gear and the circular gear, the mated teeth may have to be aligned. For example, an edge of one or more teeth of the linear gear may align with an edge of one or more teeth of the circular gear such that the edges of the teeth of the linear gear and the circular gear contact one another. The alignment may be such that there may be little or no gap and/or clearance between the teeth of the linear gear and circular gear when mated. As the motor rotates, the circular gear rotates. As mentioned above, to engage the linear gear with the circular gear, the motor may rotate the circular gear such that the edge of the teeth of the circular gear that mates with the edge of the teeth of the linear gear may align with each other. Following such engagement, rotation of the circular gear may result in a translational motion of the plunger (which is coupled to or otherwise integrated with the linear gear). For instance, rotation of the circular gear in a clockwise direction may result in movement of the plunger in a forward or distal direction and rotation of the circular gear in a counterclockwise direction may result in movement of the plunger in a backward or proximal direction within and relative to the cylindrical body, respectively. It should be readily understood that the teeth of the gears that mate are corresponding teeth that are configured to interact with one another, and thus to align to move the plunger. The directions of rotation as disclosed herein such as a clockwise direction and a counterclockwise direction may the directions when view from the drive end of the motor.

As discussed above, when the edges of the teeth of the circular gear that mate with the edges of the teeth of the linear gear align, the circular gear may be engaged with the linear gear. Accordingly, to move the plunger in a forward or distal direction (referred to as “injection” action of the plunger), first edge of the teeth of the circular gear may align with first edge of the teeth of the linear gear, thereby engaging the linear gear with the circular gear. However, to move the plunger in a backward or proximal direction (referred to as “withdrawal” action of the plunger), second opposite edge of the teeth of the circular gear may align with second opposite edge of the teeth of the linear gear, thereby engaging the linear gear with the circular gear, albeit, on opposite sides than when moving in the forward or distal direction. It should be readily understood that the teeth of the gears that align to move the plunger in the forward direction may be the same or different from the teeth of the gears that align to move the plunger in the backward direction. For example, a first tooth or set of teeth of a linear gear may align with a first tooth or set of teeth respectively of the circular gear to move the plunger in the forward direction and a second tooth or set of teeth of the linear gear may align with a second tooth or set of teeth respectively of the circular gear to move the plunger in the backward direction. In some instances, a first tooth of the linear gear may align with a first tooth of the circular gear to move the plunger in both forward and backward direction, but the edge of the (e.g., same or different) teeth that may mate and align may be opposite. For example, if the first edge of the teeth mate and align to move the plunger in a first direction, then the second opposite edge of the teeth may mate and align to move the plunger in a second, opposite direction. Accordingly, if the plunger is moved in the first direction, for example here, a forward or distal direction, and has a succeeding move in a second, opposite direction, here a backward or proximal direction, the circular and linear gears may have to be moved such that opposite edge of the teeth of the linear gear and the circular gear align. Put another way, a clearance and/or gap between the teeth may exist that may need to be accounted for. Therefore, the teeth may have to be re-aligned so that the clearance and/or gap is reduced or eliminated. This realignment may re-engage the teeth of the circular gear with the teeth of the linear gear in a manner that prepares the plunger to be moved in the opposite direction (e.g., here, backward or proximal direction).

More specifically, if a first move of the plunger is in a first direction and the succeeding move is in the same direction, the teeth of the circular gear may remain aligned with the teeth of the linear gear, thereby continuing to engage the circular gear with the linear gear. For instance, if the first move is in the forward or distal direction (e.g., emptying the syringe) also referred to as an “injecting action” and the succeeding move is also in the forward or distal direction, then the circular gear may remain engaged with the linear gear. However, if a first move of the plunger is in a first direction and the succeeding move is in a second direction opposite to the first direction, then the circular gear and/or the linear gear may need to travel (without moving the plunger) so as to re-align the teeth of the circular gear and the teeth of the linear gear and thus re-engage the gears. This movement and/or travel so as to re-align the teeth of the circular gear and the teeth of the linear gear is commonly referred to as “backlash”.

In some variations, if the pressure in the syringe pump is above a threshold value (e.g., high pressure) and/or a range of threshold values, some amount of backlash may also occur between two movements in the same direction, such as subsequent forward or backward movements. The threshold value and/or range of threshold values may be based on one or more of the design of the syringe pump, the materials used to manufacture the syringe pump, and the type of fluid in the syringe pump For example, in such variations, after the first move, the pressure in the syringe may push the plunger to the point where opposite edges of gear teeth of the circular gear may align with opposite edges of the gear teeth of the linear gear. This may cause backlash. Some or all of the backlash must be taken up to re-align the appropriate edges of the gear teeth of the circular gear with the appropriate sides of the gear teeth of the linear gear for a movement of the plunger in a forward or distal direction but the succeeding movement can begin. Although backlash may at times herein be described with reference to the linear gear and circular gear, it should be readily understood that backlash may occur between any types of gears. For example, backlash may occur between the gears in the gearbox as well, and such backlash may also be accounted for by the devices, systems, and methods described herein.

If the pressure within the cylindrical body of the syringe is above a threshold value, the pressure may cause sufficient force permitting complete engagement of the linear gear and the circular gear. However, when the pressure within the cylindrical body causes insufficient force, the teeth of the linear gear and the teeth of the circular gear teeth may have a gap (e.g., they may be floating relative to each other or may otherwise lack sufficient contact). This may cause backlash.

In some variations, an additional backlash-like effect may be associated with the enlarged end of the plunger. The enlarged end may comprise a compliant material, such as rubber, and may directly contact the inner wall of the cylindrical body. The enlarged end may provide enough force against the cylindrical body to prevent the fluid in the syringe pump from leaking. In some variations, the enlarged end (e.g., via the properties of the material of the enlarged end, such as rubber) may have some flexibility. Due to the compliance or flexible nature of the material, the plunger may have to physically move a small distance before a sufficient force is applied to the enlarged end to overcome the static friction with the cylindrical body to move any fluid into or out of the cylindrical body of the syringe pump. In order to transfer precise amounts of fluid (e.g., deliver to fluid to, or receive fluid from an expandable to control a size of the expandable member, deliver fluid to a subject), the plunger of the syringe pump must be precisely moved. Thus, the backlash due to the gears (e.g., one or more of the circular gear, the linear gear, one or more of the gears in the gearbox) and the backlash-like effect caused by the compliance of the plunger's elongated end may need to be accounted for. Accordingly, the devices, systems, and methods described herein may include one or more controllers (pump controller, device controller and/or system controller) configured to calculate the total backlash for the devices and/or system. The total backlash may be the sum of the backlash within the gears in the gearbox and the backlash between the linear gear and the circular gear as well as the movement of the linear gear required to overcome the friction between the elongated end of the plunger and the sidewall of the cylindrical body.

To reduce backlash between the linear gear and the circular gear, in some variations, the syringe and/or the controllers described herein may comprise a lever configured to maintain or assist in maintaining a center-to-center distance between the linear gear and the circular gear. It may be useful to maintain a center-to-center distance between the linear gear and circular gear to avoid backlash as well as to avoid wear and tear of the linear gear and the circular gear. If the distance between the linear gear teeth and the circular gear teeth is too large and thus a clearance exists, then for a certain number of movements of the circular gear the linear gear may translate an inaccurate distance. That is, the backlash may cause the linear gear to translate an incorrect distance for a given number of movements of the circular gear. This may manifest as imprecise fluid delivery and/or an inaccurate adjustment in the size (e.g., an inaccurate amount of inflation and/or deflation) of the expandable member. However, if the distance between the linear gear teeth and the circular gear teeth is too close, the interaction between the linear gear teeth and circular gear teeth may result in binding and excessive wear of the components. To avoid these detrimental effects on pump precision, function, and wear, in some variations, the syringe and/or controllers (e.g., system controller, device controller, and/or pump controller) described herein may comprise a lever that maintains or assists in maintaining the center-to-center distance between the linear gear and the circular gear. The lever may additionally be configured to releasably lock the syringe to the housing of the controller (e.g., system controller, device controller and/or pump controller). This may avoid unwanted movement of the syringe and may make the pump devices easier to use, especially in a field setting.

Additionally or alternatively, in some variations, additional techniques, as described in more detail herein, may be employed to compensate for backlash.

### Determining Backlash

To compensate for backlash, the controller (e.g., system controller, device controller, and/or pump controller) may be configured to determine one or more actions to reduce backlash in the devices and/or system. More specifically, the controller may be configured to determine movements that may be needed to re-engage the gears (e.g., circular gear, linear gear, gears in a gearbox, etc.), such as for example, re-aligning the appropriate sides of the gears. For instance, the controller may determine a number of additional steps (e.g., additional movement) that the motor may have to be rotated so as to re-engage the teeth of the gears (e.g., linear gear, circular gear, and/or gearbox) to inject and/or withdraw an intended amount of fluid to and/or from the cylindrical body. These additional steps and/or movements to re-engage the gears may be referred to as a backlash compensation amount. The backlash compensation amount may be determined by one or more of the following methods.

Based on Detection of Movement and/or Position of Motor or Plunger

In some variations, an amount of movement to compensate for backlash (e.g., backlash compensation amount) may be determined based on a position and/or movement of the motor. For example, the additional amount of motor movement (e.g., the number of additional steps that the motor is to be rotated to re-engage the gears (e.g., linear gear, circular gear, and/or other gears in the gearbox)) may be determined based on a position of the motor. For instance, as described above, the motor may be coupled to (e.g., optically, magnetically, or mechanically) or otherwise integrated with a sensor such as an optical encoder, a magnetic encoder, a combination thereof, and/or the like. The sensor may detect a current position of the motor and/or a change in the position of the motor between a prior point in time and the current point in time. For example, the motor may determine a motor movement amount (e.g., a total number of steps the motor may have taken) between two points in time during use (e.g., from a start of initial movement to a current point in time, from a start of a forward movement of plunger to an end of the forward movement of plunger, from a start of a backward movement of a plunger to an end of the backward movement of plunger, etc.). In some variations, based on motor movement amount (e.g., the total number of steps) between the two time points, a controller (e.g., system controller, device controller, and/or pump controller) may determine the additional motor movement needed (e.g., steps that the motor may have to take) to re-engage the teeth of the gears. In one variation, the controller may identify a volume of fluid to be transferred in and/or out of the syringe (e.g., based on desired amounts of fluid to be delivered, desired size of an expandable member (which, in variations used with a blood flow control device, may in turn be based on one or more blood pressure measurements). Based on the volume of the fluid and the total number of steps the motor has taken (e.g., current position of motor), the controller may determine an additional motor movement needed (e.g., additional steps that the motor may have to take) to re-engage the gears. While described above with respect to the motor, it should be appreciated that in some variations, the amount of movement to compensate for backlash may be determined based on a position and/or movement of another component of the pump, such as the circular gear or the linear gear.

Additionally or alternatively, a backlash compensation amount may be determined based on a position and/or movement of the plunger relative to the cylindrical body. For example, the additional motor movement needed (e.g., number of additional steps that the motor is to be rotated) to re-engage the gears may be determined based on a position and/or movement of the plunger relative to the cylindrical body. As discussed above, the syringe may include one or more sensors (e.g., optical sensors, magnetic sensors, and/or linear potentiometers) configured to detect the position of the plunger within the cylindrical body and/or to track movement of the plunger. The position of the plunger may be used to determine a backlash compensation amount that may be needed to re-engage the circular gear and the linear gear.

For example, if a move of 100 microliters of fluid is intended in a first direction and the overall gear ratio is 1 step per microliter, the controller may actuate the motor (e.g., stepper motor). The motor may be actuated until a movement of the plunger is detected (e.g., via one or more sensors). Movement of the plunger may indicate that the appropriate edges of the teeth of the linear gear and the circular gear are aligned, thereby engaging the linear gear and the circular gear for movement in the desired direction. For instance, movement of the plunger in a forward or distal direction may indicate that first edge of teeth of the circular gear may be aligned with first edge of teeth of the linear gear. Movement of the plunger in a backward or proximal direction may indicate that second opposite edge of teeth of the circular gear may be aligned with second opposite edge of teeth of the linear gear. The controller may record the movement of the motor until a movement of the plunger is detected. This may be the backlash compensation amount. Once movement of the plunger is detected (and thus the teeth of the linear gear have engaged the teeth of the circular gear), the controller may actuate the motor so that the motor moves the (additional) amount necessary to transfer the 100 microliters of fluid. This may be the fluid transfer movement amount.

### Based on Measurement of Pressure

In some variations, an amount of movement to compensate for backlash (e.g., backlash compensation amount) may be determined based on a measurement of pressure. As discussed above, to compensate for backlash, a backlash compensation amount may be determined based on a position and/or movement of the plunger relative to the cylindrical body. In some variations, the position and/or movement of the plunger relative to the cylindrical body may be indirectly measured based on a measurement of pressure within the cylindrical body of the syringe and/or a measurement of pressure within an expandable member fluidly coupled to the syringe (in variations in which the syringe is used with a blood flow control device or other treatment device with an expandable member). More specifically, in some variations, an additional movement of the plunger to re-engage gears within the system (e.g., the circular gear and the linear gear, gears in a gearbox) may be based on the amount of pressure inside the cylindrical body of the syringe and/or the expandable member. For example, if the first move of the plunger is an injection action (forward direction) and the next move of the plunger is a withdrawing action (backward direction), then the amount of movement of the plunger to re-engage the gears (e.g., the linear gear and the circular gear, gears in gearbox) may be dependent on the pressure inside the cylindrical body of the syringe. A higher pressure may actively push the plunger in the withdrawing or backward direction. However, the high pressure in the cylindrical body of the syringe may move the plunger so that the mated teeth of the gears have reduced clearance between them such that the gears are closer to being engaged or are engaged to gear the plunger for a forward movement. More specifically, the high pressure in the cylindrical body of the syringe may move the plunger so that the gears are aligned such that they are closer to being engaged or are engaged to gear the plunger to move forward. Therefore, if the pressure in the syringe pump is high, smaller movements may be enough to re-engage the gears so that the plunger is geared to perform the injecting action. For example, smaller movements of the motor may re-engage the gears such that the gears may allow the plunger to perform the injecting action.

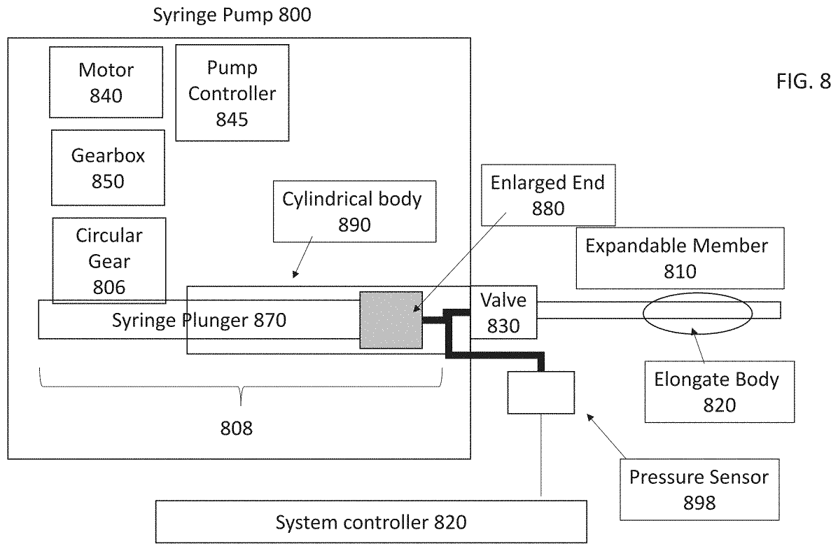

FIG. 8 shows an exemplary variation of a system (e.g., a blood flow control system described in further detail below) for determining additional movement needed to compensate for backlash (e.g., a backlash compensation amount) via measurement of pressure. Shown there is a syringe pump 800 comprising a syringe 808 with a cylindrical body 890 and a plunger 870 with an enlarged end 880 slidably positioned within the cylindrical body 890. The syringe pump 800 may also include a motor 840, a circular gear 806, a gearbox 850, and a pump controller 845. In the variation depicted here, an elongate body 820 may be coupled to the syringe 808. The distal tip of the elongate body 820 may include an expandable member 810. The syringe 808 may be used to inflate and/or deflate the expandable member 810. As seen in FIG. 8, a pressure sensor 898 may be positioned near or otherwise in fluid communication with the distal tip of the syringe pump and may be configured to measure pressures indicative of pressures inside the syringe. In some variations, the pressure sensor 898 may measure a pressure inside the expandable member 810 (i.e., it may be an expandable member pressure sensor), which may be indicative of a pressure inside the syringe. While depicted outside the syringe pump 800 and the pump controller 845, it should be appreciated that in some variations, the pressure sensor 898 may be contained within or otherwise carried by the controller housing. The pressure sensor 898 may measure the pressure in the fluid path between the syringe pump 808 and an optional valve 830. In some variations, the pressure sensor 898 may be communicatively coupled to a controller (e.g., system controller, device controller, or pump controller) that receives the pressure data from the pressure sensor 898 and analyzes the pressure data.

When the plunger 870 moves, the pressure in the fluid pathway between the syringe pump 808 and the valve 830 changes. The pressure in the fluid pathway may be indicative of the pressure inside the cylindrical body 890 and the pressure inside the expandable member 810. Put differently, the pressure inside the cylindrical body 890 may be the same as the pressure inside the expandable member 810 which may be the same as the pressure in the fluid pathway. This is because the syringe 808 is fluidly coupled to the expandable member 810 via the fluid path. The expandable member pressure and/or pressure in the cylindrical body 890 may be indicative of the amount of inflation and deflation of the expandable member and consequently the amount of translation of the plunger relative to the cylindrical body.

This pressure may be measured to calculate a backlash compensation amount. For instance, the additional motor movement (e.g., number of additional steps that the motor is to be rotated) may be linearly dependent on the pressure inside the cylindrical body of the syringe. Accordingly, the number of additional movement (e.g., steps) may be N=mp+c; where N is the additional movement (e.g., steps), p is the pressure in the cylindrical body of the syringe, and m and c are constants.

In some variations, a memory of the controller may store a lookup table to determine the amount of additional movement (e.g., number of additional steps) to compensate for backlash. For example, the lookup table may include the amount of additional movement (e.g., number of additional steps) for a plurality of ranges of pressures in the cylindrical body of the syringe. In some variations, the lookup table may include a volume of fluid to be transferred in or out of the syringe for a plurality of ranges of pressures in the cylindrical body of the syringe. The controller may determine the amount of additional motor movement (e.g., a number of additional steps that the motor is to be rotated) based on the volume of the fluid to be transferred identified from the lookup table. Table 1 is an exemplary lookup table for calculating a backlash compensation amount based on the pressure inside the syringe. As seen in Table 1, the number of additional steps that the motor is to be rotated may be greater for lower pressures inside the syringe as opposed to the number of additional steps that the motor is to be rotated for higher pressures. For example, in exemplary Table 1, for pressures that are greater than 175 mmHg, the amount of additional motor movement to compensate for backlash may be the amount of steps needed by the motor to transfer 20 uL into the syringe. For a pressure range between 150 mmHg and 175 mmHg, the amount of additional steps the motor may have to take to compensate for backlash may be the amount of steps needed by the motor to transfer 50 uL into the syringe. For a pressure range between 125 mmHg and 150 mmHg, the amount of additional steps the motor may have to take to compensate for backlash may be the amount of steps needed by the motor to transfer 125 uL into the syringe.

TABLE 1

| | Amount of Additional Fluid to Request for |
|---|---|
| Syringe/Balloon | Gear-Loading (essentially additional motor- |
| Pressure Range | steps) |
| >175 mmHg | 20 uL |
| Between 150 to 175 mmHg | 50 uL |
| Between 125 to 150 mmHg | 125 uL |
| <125 mmHg | 170 uL |

While FIG. 8 depicts a variation of a syringe 808 (and syringe pump 800) fluidly coupled to an elongate body with an expandable member, in some variations, the syringe pump 800 may be used independently of an elongate body, such as, for example, to deliver fluids directly to a subject. In these variations, a pressure sensor may be positioned on or otherwise in a fluid pathway between the subject and the syringe pump, and this pressure data may be indicative of the pressure inside of the cylindrical body of the syringe 808 and may be utilized in a similar manner as discussed above with respect to FIG. 8 to compensate for backlash or the backlash-like effect described herein in the syringe pump.

Turning back to the system depicted in FIG. 8 comprising a syringe pump 800 and an elongate body with an expandable member, in some variations, the elongate body with the expandable member may be or form part of a blood flow control device. As described in more detail herein, the blood flow control device may be used to cause a change in blood flow in a blood vessel, and thus a subject's blood pressure. More specifically, a change in the size (e.g., volume) of the expandable member of the blood flow control device positioned with a blood vessel may partially or fully occlude the blood vessel, thereby causing a change in blood pressure. The change in blood pressure may be monitored using one or more sensors positioned on the elongate body. A change in pressure inside the expandable member may be monitored using an expandable member sensor (e.g., an expandable member sensor positioned in a controller).

Monitoring pressure inside the expandable member (e.g., using an expandable member sensor) and blood pressure of the subject may provide information on the amount of inflation and deflation of the expandable member. The expandable member pressure may be received in the form of waveforms. The controller may identify spikes (e.g., instantaneous ascent and/or instantaneous descent) in the expandable member pressure waveforms. Differentiating between the spikes in the expandable member pressure may help identify the amount of inflation and deflation of the expandable member. For instance, differentiating between the spikes may help identify whether the plunger has translated to either inject fluid and/or withdraw fluid from the expandable member. The change in expandable member sensor pressure may also be used to calculate a backlash compensation amount.

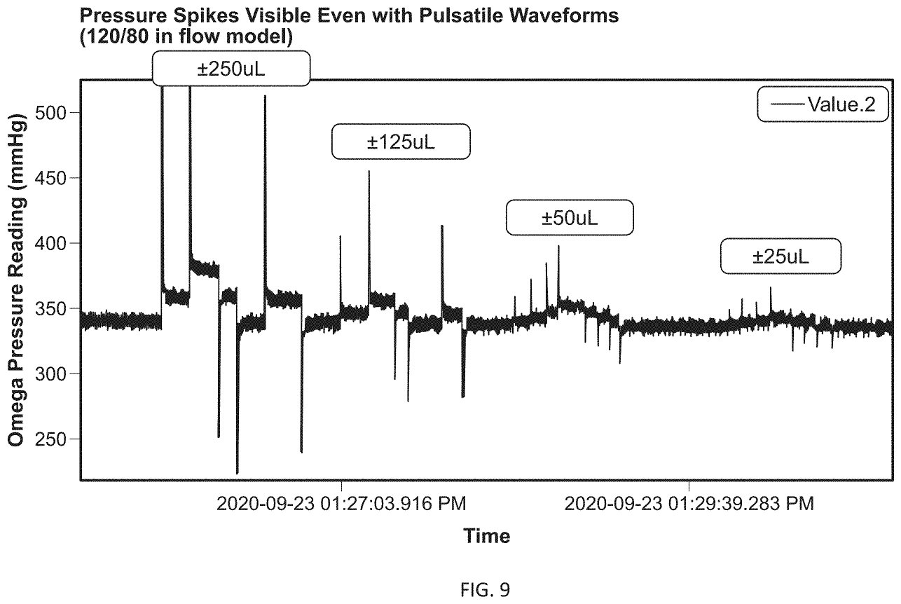

Referring to FIG. 9, shown there is a plot depicting expandable membrane pressure waveforms. The plot in FIG. 9 shows that movements of 100-200 μL of fluid may be distinguishable from the baseline pressure waveforms in the expandable membrane pressure caused by the blood pressure exerting an effect on the external surface of the expandable membrane. When the plunger moves forward (emptying the syringe) the pressure at the pressure sensor 898 increases rapidly (e.g., instantaneously ascends) and then drops rapidly (e.g., instantaneously descends) as the fluid moves to the expandable member. Therefore, the controller may receive a spike in the expandable member pressure. Similarly, when the syringe is moved backwards (filling the syringe) the pressure at the pressure sensor 898 decreases rapidly and then increases back to the new equilibrium pressure. This rate of change in pressure may be analyzed by the controller. The analyzed rate of change may be used to calculate a backlash compensation amount and/or when the backlash compensation has been completed.

The controller may receive sensor data from the pressure sensor at a faster rate (e.g., at least two times faster, at least 2.5 times fast, at least 3 times faster, or more) than the rate at which the expandable member pressure changes, and may then calculate the rate of change in the expandable member pressure. When the rate of change of expandable member pressure exceeds a threshold and/or the amplitude of the spike in the expandable member pressure waveforms exceeds a threshold, this may indicate that the plunger is moving. This change in expandable member pressure may be used to calculate a backlash compensation amount and/or when the compensation has been completed.

### Based on a Measurement of Motor Torque

As discussed above, the movement needed to compensate for backlash may be determined based on a position and/or movement of the plunger within the cylindrical body. In some variations, the position and/or movement of the plunger relative to the cylindrical body may be indirectly measured based on a measurement of torque on the motor. For example, when rotating the gears to compensate for backlash, the torque on the motor may be lower than when rotating the gears to move the plunger. By identifying whether the torque on the motor is below a threshold, the controller (e.g., system controller, device controller, and/or pump controller) may determine whether the motor is rotating the gears to compensate for backlash or rotating the gears to transfer fluid.

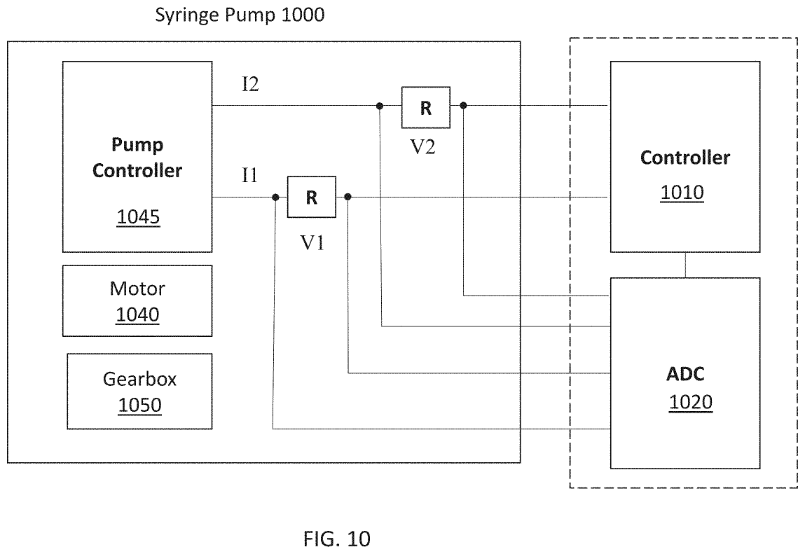

FIG. 10 shows an exemplary variation of a syringe pump 1000 configured to indirectly detect movement of the plunger via torque estimation. In FIG. 10, a pump controller may comprise a motor 1040 and a gearbox 1050. The pump controller 1045 may comprise a circuit that may drive currents (e.g., I1 and I2) to two sets of windings in the motor 1040. The controller 1045 may identify that the maximum current has been reached by driving that current through a precision resistor (e.g., shown as R in FIG. 10) and measuring the voltage across that resistor. Two separate resistors may be employed, one for each of the two windings on the motor. When the current hits a threshold value, the pump controller 1045 may stop driving current to the motor 1040, and that completes the “step” of the motor 1040.

In FIG. 10, a controller 1010 may comprise an analog-to-digital converter, (ADC) 1020 that may measure the voltage across the resistors. In some variations, the ADC 1020 may form part of the pump controller 1045, while in other variations, such as the one depicted in FIG. 10, the pump controller 1045 may be communicatively coupled to the ADC 1020. The pump controller 1010 may receive the output signals from the ADC 1020 and may compare an ascent and/or increase in the voltages from one step of the motor to the next step of the motor. The increase in the voltage varies as the torque on the motor changes. The torque on the motor is lower when rotating the gears through the backlash than when actually moving the plunger and transferring fluid. By detecting the change in the voltage gradient, the syringe pump and/or pump controller may determine when the backlash has been compensated for. In some variations, the controller may comprise a motor current measurement unit to measure the current due to the change in the voltage gradient. The measured current may be used to determine when the backlash has to be compensated for and/or may be used to determine a backlash compensation amount as discussed below.

### Automatically Compensating for Backlash

To compensate for backlash, in some variations, a controller (e.g., system controller, device controller, and/or pump controller) may predispose and/or bias a gear (e.g., the circular gear) in a particular direction, referred to herein as a biasing direction. When biased in a particular direction, the position of the gear may be maintained, or the gear may be moved to reassume a position, priming the plunger for immediate movement (when so instructed) in a particular direction, regardless of the prior movement (including direction of movement) of the gear. For example, if a gear is biased in a clockwise rotational direction (corresponding to a forward or distal direction for the plunger), after a movement in the counterclockwise rotational direction, the teeth of the gear may be re-aligned with corresponding teeth of another gear such that the plunger is ready (or geared) for immediate movement in the forward or distal direction (i.e., there is no gap or clearance between the mating teeth of the gears for movement in the forward direction). Continuing with this example, after movement in the clockwise rotational direction, the teeth of the gear may remain aligned with corresponding teeth of the other gear such that the plunger remains ready for immediate movement in the forward direction when so instructed. In this manner, the gear may be biased in the clockwise rotational direction or the forward linear direction. Thus, biasing the gear towards rotating in the biasing rotational direction may cause the plunger to be geared towards translating in a biasing linear direction. In some variations, the biasing rotational direction may be a clockwise direction, while in other variations, the biasing rotational direction may be a counterclockwise direction. Similarly, in some variations, the biasing linear direction may be a forward or distal direction, while in other variations, the biasing linear direction may be a backward or proximal direction.

While the circular gear may be biased in either direction, biasing the circular gear so that the plunger is geared to move in the forward direction may be advantageous. For example, in variations in which the syringe pump is fluidly coupled to the expandable member, gearing the plunger to move in the forward direction may result in ensuring that the expandable member can inflate quickly. This in turn may be useful in occluding a blood vessel of a subject (e.g., blood vessel into which the expandable member is advanced and positioned) and in preventing bleeding. In a similar manner, in variations in which the syringe pump delivers fluid to a patient, gearing the plunger to move in the forward direction may result in faster delivery of medication. Furthermore, as discussed above, the pressure in the cylindrical body of the syringe may actively push the plunger in the backward (filling the syringe) direction. Accordingly, if in a first move the plunger performs a withdrawing action (e.g., backward direction) and if the plunger is to perform an injection action (e.g., forward direction) in the succeeding move, then the circular gear may not only have to compensate for backlash due to the gears but may also have to compensate for slop due to pressure inside the syringe. This in turn may require additional motor movement (e.g., the motor may need to rotate an additional number of times), thereby consuming excess energy. Therefore, the circular gear may be biased so that the plunger is geared to move in the forward direction.

As discussed above, in some variations, a gear may be biased towards a biasing direction, thus gearing the plunger for movement in the biasing linear direction. In these variations, a controller may be configured to determine a backlash compensation amount based on whether the next desired movement of the plunger is in the biasing linear direction (and thus the next desired movement of the circular gear is in the biasing rotational direction), or opposite the biasing linear direction (or opposite the biasing rotational direction). More specifically, and as will be discussed in more detail below, the controller may be configured to determine a backlash compensation amount when the next desired movement of the plunger is in a linear direction opposite the biasing linear direction only. Put another way, when the next desired movement of the plunger is in the same linear direction as the biasing linear direction, there is no need to determine a backlash compensation amount for that desired movement (because the gears are already positioned to move in that direction by virtue of the biasing). However, when the next desired movement is opposite the biasing linear direction, the controller may be configured to determine a backlash compensation amount.

As discussed above, when the next desired movement of the plunger is in the same linear direction as the biasing linear direction, there is no need to determine a backlash compensation amount for that desired movement. In such variations, after transferring fluid to or from the syringe, the gears may be maintained in a predetermined biasing direction. That is, if after transferring fluid, the next desired movement of the plunger is in the same linear direction as the biasing linear direction, then the gears may be maintained in the biasing direction such that the plunger may be geared for movement in the biasing linear direction. When the desired movement of the plunger is in the biasing linear direction, a controller (e.g., system controller, device controller, and/or pump controller) may be configured to determine the amount of movement (e.g., of the circular gear, linear gear, motor) needed to move a component of the syringe pump (e.g., motor, circular gear, linear gear, plunger, and/or other gears in gearbox) by a desired amount in the biasing direction. For example, the controller may determine the number of rotations of the circular gear to move the plunger in the biasing linear direction by the desired amount. Additionally or alternatively, the controller may determine the amount of motor movement (e.g., a number of motor steps) required to move the plunger in the biasing linear direction by the desired amount. The controller may rotate the circular gear in the biasing rotational direction by the amount needed to achieve movement of the plunger in the desired amount (i.e., a determined amount). Thus, moving the circular gear by the determined amount in the biasing rotational direction may cause the linear gear, and thus the plunger, to move in the biasing linear direction by the desired amount, thus transferring a desired amount of fluid (e.g., medication) to a patient and/or transferring a desired amount of fluid into/out of an expandable member.

More specifically, for example, if the initial desired movement of the plunger is in the biasing linear direction, which, in this example, is in a forward direction, and the next desired movement is in the forward direction, the controller may determine the amount of movement needed to move the plunger in the forward direction by the desired amount. The controller may then move the circular gear in a clockwise direction by the determined amount. Moving the circular gear in the clockwise direction by the determined amount causes the linear gear and therefore the plunger to move in the forward direction by the desired amount. Thus, no backlash compensation amount is needed to achieve the desired movement of the plunger in the desired amount.

If however, the desired movement is in a direction opposite the biasing linear direction, the controller may be configured to determine a backlash compensation amount. The backlash compensation amount may account for various forms of backlash. For example, in some variations, the backlash compensation amount may include one or more of a backlash compensation amount due to backlash between the circular gear and the linear gear, a backlash compensation amount due to backlash in a gearbox, and a backlash compensation amount due to plunger compliance.

As discussed above, when the desired movement is in a direction opposite to the biasing linear direction, the controller may determine a backlash compensation amount. To move the plunger in the direction opposite to the biasing linear direction to transfer a desired amount of fluid, the controller may be configured to additionally determine a fluid transfer movement amount that corresponds to the desired amount of fluid to be delivered from or received within the syringe and/or the desired change in size (e.g., volume) of an expandable member. The controller may be further configured to determine a total movement amount for a component of the syringe pump (e.g., motor, circular gear, linear gear, plunger, gears in the gearbox, etc.) in the direction opposite to the biasing linear direction, which may be the sum of the backlash compensation amount and the fluid transfer movement amount. The total movement amount in the direction opposite to the biasing linear direction may be the amount of movement of the component, such as for example, the amount of movement of the motor (e.g., the number of steps the motor may have to take), circular gear, linear gear, other gears in gearbox, etc. to move the plunger in the direction opposite to the biasing linear direction to transfer the desired amount of fluid. In some variations, when the desired movement is opposite to the biasing linear direction, the controller may determine an overall movement amount. The overall movement amount may be the total movement amount in the direction opposite to the biasing direction and a biasing movement amount (described below) in the biasing direction. The total movement amount may transfer the desired amount of fluid and compensate for backlash, while, in these variations, the overall movement amount may result in both transfer of the desired amount of fluid (while compensating for backlash) and biasing of the gears in the biasing direction as further described below.

For example, the controller may determine the total movement amount for the motor (e.g., number of steps the motor may have to take) in the direction opposite to the biasing direction. In some variations, the controller may also determine an overall movement amount for the motor. The controller may move the motor such that the circular gear rotates in a direction counter to the biasing rotational direction the total movement amount. This may move the plunger in the direction opposite the biasing linear direction such that the desired amount of fluid is transferred and the backlash is compensated for. The movement in the biasing direction is discussed below.

As discussed above, in some variations, the controller may bias the gear in the biasing direction. That is, the controller may be configured to bias the circular gear in the biasing rotational direction. Accordingly, after the plunger has moved in the direction opposite the biasing linear direction to transfer the desired amount of fluid, the controller may re-engage the linear gear and the circular gear. To re-engage the linear gear and the circular gear, the controller may determine a biasing movement amount. For example, the controller may determine an amount of movement of a component, such as for example, amount of movement of the motor (e.g., number of steps the motor may have to take), circular gear, linear gear, other gears in gearbox, etc. that corresponds to the biasing movement amount. As mentioned above, the controller may determine an overall movement amount which may be the total movement amount in the direction opposite the biasing direction and the biasing movement amount in the biasing direction. Accordingly, after moving the motor in the direction opposite to the biasing direction, to re-engage the linear gear and the circular gear, the controller may move the motor such that the circular gear rotates in the biasing rotational direction. This may re-engage the circular gear with the linear gear, thereby biasing the circular gear in the biasing direction and gearing the plunger for movement in the biasing linear direction. In some variations, the biasing movement amount may be the same as the backlash compensation amount (albeit in opposite directions). More specifically, the absolute value of the biasing movement amount may be the same as the absolute value of the backlash compensation amount. In some variations, the absolute value of the biasing movement amount may be different from the absolute value of the backlash compensation amount. In such variations, the controller may determine the biasing movement amount based on one or more factors such as for example, pressure inside the syringe, pressure inside an expandable member (in variations in which the syringe pump is coupled to a blood flow control device), a position of the motor, a position of the circular gear, a position of the plunger, an amount of time between two consecutive movements of the plunger, a combination thereof, and the like. In such variations, the controller may receive sensor data from one or more sensors described herein to determine the biasing movement amount.

It should be readily understood when the controller moves the component such as for example, motor, circular gear, linear gear, other gears in gearbox, etc. in the biasing direction to re-engage the gears, the plunger may remain substantially stationary. Put differently, the controller may re-engage the gears without materially moving the plunger. For instance, to the extent there is any movement of the plunger, the movement of the plunger may be insignificant such that it may not cause transfer of a physiologically significant amount of fluid. For example, to the extent there is any movement of the plunger, the movement may cause only at most only a few μl (e.g., 1 μl or less) of fluid to be transferred in or out of the syringe.

In some variations, the backlash compensation amount and/or the biasing movement amount may be a determined movement amount that may be determined by the controller before moving a component of the syringe pump such as for example, motor, circular gear, linear gear, other gears in gearbox, etc. In some variations, the backlash compensation amount and/or the biasing movement amount may be determined based on factors described herein such as for example, pressure inside the syringe, pressure inside an expandable member (in variations in which the syringe pump is coupled to a blood flow control device), a position of the motor, a position of the circular gear, a position of the plunger, an amount of time between two consecutive movements of the plunger, a combination thereof, and the like.

As an example, if an initial movement of the plunger is in a forward direction (e.g., biasing linear direction), the controller may move the circular gear in a clockwise direction (e.g., biasing rotational direction), thereby moving the plunger forward. Now, if the next desired movement of the plunger is in a backward direction (e.g., opposite the biasing linear direction) by a desired amount, then the controller may determine a backlash compensation amount and a fluid transfer movement amount to move the plunger in the backward direction by the desired amount. The controller may calculate a total movement amount (e.g., sum of the backlash compensation amount and the fluid transfer movement amount) to move the plunger in the backward direction. The controller, in some variations, may determine an overall movement amount. The overall movement amount may be the total movement amount in the backward direction and the biasing movement amount in the forward direction.

The controller may move the motor by the total movement amount such that the circular gear rotates in the counterclockwise direction (e.g., counter to the biasing rotational direction). Moving the circular gear in the counterclockwise direction by moving the motor by the total movement amount may move the plunger in the backward direction (e.g., opposite to the linear biasing direction) by the desired amount. After moving the plunger in the backward direction by the desired amount, the controller may move the motor by the biasing movement amount such that the circular gear moves in the clockwise direction, thereby biasing the circular gear in the clockwise direction and gearing the plunger towards the forward direction.

In some variations, consider that an initial movement of the plunger is against a biasing linear direction and that a succeeding movement of the plunger is also to be against the biasing linear direction by a desired amount. After the initial movement against a biasing linear direction, the circular gears are biased towards the biasing rotational direction and the plunger is geared towards the biasing linear direction as discussed above. Therefore, to make the succeeding move against the biasing linear direction the controller may still have to compensate for backlash. Accordingly, the controller may calculate a backlash compensation amount and a fluid transfer movement amount. The controller may calculate the total movement amount from the backlash compensation amount and the fluid transfer movement amount. The controller may also calculate the biasing movement amount, which in some variations may be the same as backlash compensation amount. The controller may calculate the overall movement amount which may be the total movement amount against the biasing direction and the biasing movement amount in the biasing direction. The controller may first move the motor by the total movement amount such that the circular gear rotates in a direction counter to the biasing rotational direction. That is, the controller may rotate the circular gear in the direction counter to the biasing rotational direction such that the teeth of the circular gear may re-align with the teeth of the linear gear, thereby engaging the linear gear and the circular gear and moving the plunger against the biasing linear direction by the desired amount. After the plunger has moved against the biasing linear direction by the desired amount, the controller may move the motor by the biasing movement amount to rotate the circular gear in the biasing rotational direction. This may result in biasing the circular gear in the biasing rotational direction and gearing the linear gear towards the biasing linear direction. At this point, the controller has moved the motor the overall movement amount.

As a non-limiting example, consider that the motor has a step-to-uL ratio of 1:1. That is, for every step the motor takes, 1 uL of fluid is transferred to or from the syringe. In this example, a determined amount of rotation for the motor (e.g., the number of steps that a motor has to take) to compensate for backlash may be 50 steps. Additionally, in this example, the biasing linear direction is forward and biasing rotation direction is clockwise. If in the first move, the plunger is to be moved in a first linear direction, and in the same direction as the linear biasing direction, in this example, a forward direction, to deliver 100 uL of fluid, then the controller may rotate the motor by 100 steps in the first rotational direction, here, clockwise, so that the circular gear rotates by 100 steps in the first rotational direction (also the biasing rotational direction), the clockwise direction. The rotation of the motor by 100 steps in the first rotational direction may cause the plunger to translate in a forward direction to deliver 100 uL of the fluid. At this point, the circular gear remains biased in the clockwise direction and the plunger remains geared in the forward direction. If, in the second move, the plunger is to be moved in a second, opposite linear direction (opposite the biasing linear direction), here the backward direction, to fill the syringe pump with 100 uL of fluid, then the controller may rotate the motor by 150 steps in the second rotational direction (opposite the biasing rotational direction). This is because the determined amount (e.g., steps) for compensating for backlash is 50 steps and the fluid delivery movement amount to transfer 100 uL of fluid is 100 steps. Put differently, 50 steps of the 150 steps may re-engage the circular gear and the linear gear in the counterclockwise direction (e.g., determined amount for backlash compensation) and the remaining 100 steps may fill the syringe pump with 100 uL of fluid (e.g., predetermined fill amount). After this, the controller may automatically rotate the motor by 50 steps (e.g., predetermined steps for backlash compensation) in the first rotational direction to re-engage the circular gear and the linear gear in the rotational biasing direction, here clockwise direction. At this point, the circular gear is biased in the clockwise direction and the plunger is geared towards the forward direction.

In some variations, to compensate for backlash, the controller may not bias the circular gear in any specific direction. Instead, when the direction of fluid transfer is changed, the controller may calculate a backlash compensation amount and re-engage the gears before transferring the fluid. Put differently, if the plunger has performed an injection action (in the forward or distal direction) and is to perform a withdrawal action (in the backward or proximal direction) by a desired amount in the next move, the controller may calculate a backlash compensation amount to re-engage the gears in the counterclockwise direction and a movement needed to perform the withdrawal action by the desired amount. Accordingly, the controller may move the motor by the total movement amount such that circular gear rotates in the counterclockwise direction. The total movement amount may be the sum of the backlash compensation amount and the fluid transfer movement amount (the movement needed to perform the withdrawal action by the desired amount). The plunger may therefore move by the desired amount in the backward direction. After this movement, if the plunger has to perform an injection action by a second desired amount, the controller may determine a second backlash compensation amount (which may or may not be the same value as the first backlash compensation amount) to re-engage the gears in the clockwise direction and a second fluid transfer movement amount (the movement needed to perform the injection action by the second desired amount). Accordingly, the controller may move the circular gear by a total movement amount in the clockwise direction. The total movement amount may be the sum of the backlash compensation amount and the fluid transfer movement amount. The plunger may therefore move by the second desired amount in the forward direction.

In some variations, a movement to move the plunger forward by a desired amount, a movement to move the plunger backward by a desired amount, and backlash compensation amount to compensate for backlash may be determined based on a lookup table. For instance, the lookup table may associate the amount of rotation of the circular gear and/or the motor with various positions of the plunger and/or other components in the syringe pump (e.g., the position of the motor) (e.g., various intended amount of fluid to be delivered, intended amount of fluid to be withdrawn, etc.). Depending on the position of the plunger, the lookup table may provide the movement to move the plunger forward by a desired amount, the movement to move the plunger backward by a desired amount, and/or the backlash compensation amount to compensate for backlash that may be needed for the circular gear. In some variations, one or more of the movement to move the plunger forward by the desired amount, the movement to move the plunger backward by the desired amount, and/or backlash compensation amount to compensate for backlash may be predetermined based on one or more of the gear ratio of the gearbox, module, step angle of the stepper motor, gear ratio between the linear gear and the circular gear, pressure within the expandable member, and the like.

As mentioned above, in some variations, to compensate for backlash the controller may bias the gears in a biasing direction. In other variations, to compensate for backlash the controller may not bias the gears. In the variations in which the controller may bias the gears, if the desired movement of the plunger is to transfer a desired amount of fluid in the biasing linear direction, then the total movement for the motor may correspond to a fluid transfer movement amount. The controller may move the motor by the fluid transfer movement amount to rotate the circular gear in the biasing rotation direction and move the plunger in the biasing linear direction to transfer the desired amount of fluid. However, if the desired movement of the plunger is to transfer a desired amount of fluid in a direction opposite to the biasing linear direction, then the total movement of the motor may correspond to backlash compensation amount (counter to the biasing rotational direction) plus the fluid transfer movement amount (counter to the biasing rotational direction) plus the biasing movement amount (in the biasing rotation direction). Put differently, the controller may move the motor by the total amount (e.g., fluid transfer movement amount plus the backlash compensation amount) to rotate the circular gear counter to the biasing rotational direction, thereby moving the plunger opposite to the biasing linear direction such that the desired amount of fluid is transferred. Then, the controller may move the motor by the biasing movement amount (which may be the same as the backlash compensation amount) such that the circular gear may rotate in the biasing rotational direction, thereby re-engaging the circular gear with the linear gear.

In variations in which the controller may not bias the gears, if the desired movement of the plunger is in the same direction as the preceding movement of plunger, then the total movement for the motor may correspond to the fluid transfer movement amount. The controller may move the motor by the fluid transfer movement amount to move the plunger to transfer the desired amount of fluid. However, if the desired movement of the plunger is in a direction opposite to the direction as the preceding movement of the plunger, then the total movement for the motor may correspond to sum of the backlash compensation amount and the fluid transfer movement amount. The controller may move the motor by the total movement amount to move the plunger to transfer the desired amount of fluid.

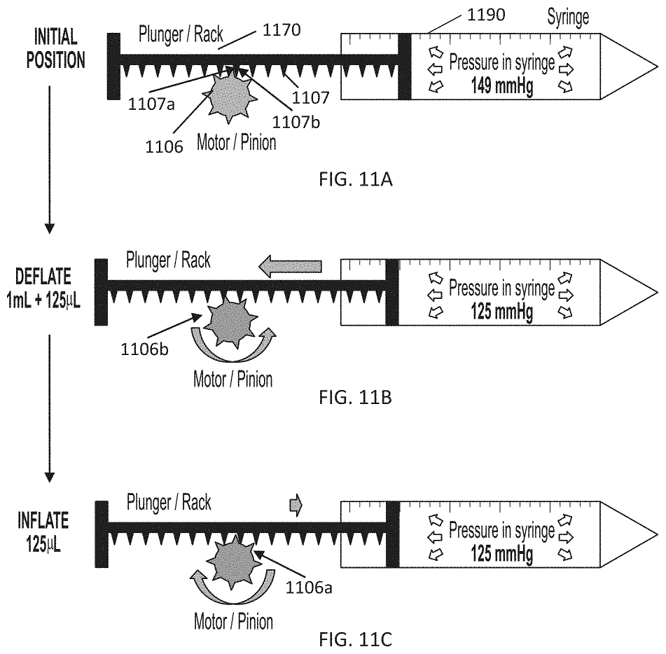

In some variations, the determined amount of movement for backlash compensation may be determined based on a movement of the plunger, the amount of pressure in the syringe pump and/or the expandable member, and/or a measurement of torque on the motor as described above. FIGS. 11A-11C depict an exemplary variation of a movement of a circular gear and a linear gear based on the pressure inside the syringe pump and the biasing of the gears such that the plunger is geared in the forward direction. FIG. 11A depicts an initial position of the plunger 1170. In this position, the surface of a tooth 1106a of the circular gear 1106 is aligned with the surface of a tooth 1107a of the linear gear 1107 such that the gears are biased to move the plunger in the forward direction. That is, the teeth of the linear gear 1107 and the circular gear 1106 are aligned such that the linear gear and circular gear are engaged so that the plunger is already positioned (geared) to perform the injection action (positioned to move in the forward direction). The pressure inside the syringe pump is shown to be 149 mmHg. In FIG. 11B, the plunger is moved in the backward direction to fill 1 mL of fluid into the cylindrical body, which in variations in which the syringe pump is fluidly coupled to an expandable member, results in deflating the expandable member. The pressure inside the syringe pump is shown to be 125 mmHg. To move the plunger in the backward direction to fill 1 mL of fluid, the controller may automatically determine a backlash compensation amount. For example, the controller may determine an amount to rotate the circular gear and/or the motor so as to align side 1106b of the circular gear 1106 with side 1107b of the linear gear 1107. That is, the controller may determine an amount to rotate the circular gear and/or the motor to re-engage the circular gear 1106 and the linear gear 1107 to perform withdrawal action (positioned to move in backward direction).

As discussed above, this backlash compensation amount may be based on the pressure inside the syringe. Higher syringe pressure may cause the backlash compensation amount to be smaller. In some variations, the backlash compensation amount may be determined based on a lookup table. For instance, the lookup table may provide the backlash compensation amount for various syringe pressures. In FIG. 11B, the pressure inside the cylindrical body is 125 mmHg. Based on table 1 described above, the backlash compensation amount is the additional amount of steps needed by the motor to transfer 125 uL of fluid into the syringe.

The controller may separately also determine the fluid transfer movement amount, here, for example, the amount of movement needed to transfer 1 mL of fluid. That is, the controller may determine the amount of motor movement (e.g., number of steps) needed to transfer 1 mL of fluid to the syringe. The controller may rotate the circular gear and the motor by a summation of the backlash compensation amount and fluid transfer movement amount to fill the syringe pump with the intended amount of fluid. In this example, the predetermined backlash amount is 125 uL and the predetermined fill amount is 1 mL. Therefore, to fill the syringe with 1 mL of fluid, the controller may rotate the circular gear by 1 mL+125 uL such that the plunger moves in the backward direction to fill the syringe with 1 mL of fluid. In variations in which the controller is configured to bias the circular gear such that the plunger is geared towards the forward direction, the controller may additionally rotate the motor and/or the circular gear by the predetermined backlash compensation amount 125 uL so that the plunger is biased in the forward direction, for example, as seen in FIG. 11C.

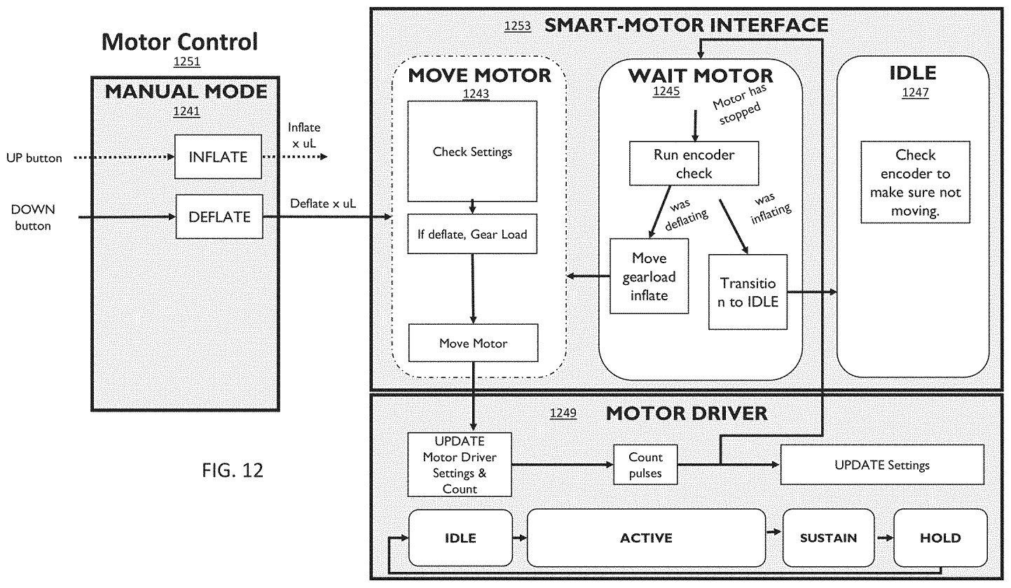

FIG. 12 depicts an exemplary variation of states that a controller (e.g., system controller, device controller, pump controller) may execute to move the plunger of the syringe in the syringe pump when the syringe pump is used with a blood flow control device or other device with an expandable member. For instance, FIG. 12 may depict the states of a controller when the variation for compensating for backlash includes biasing one or more gears in a biasing direction. The controller may include a manual mode motor control mode 1251 configured to receive input from a user via a user interface and a smart-motor interface mode 1253 during which the controller may be configured to automatically adjust expandable member volume. The motor control mode 1251 may be implemented in response to receiving one or more inputs from a user via a user interface of the controller and/or via input controls of the controller. For example, the user interface may include widgets, capacitive buttons, resistive buttons, capacitive panels, resistive panels, a combination thereof, and/or the like. The user may press and/or touch a button to implement the motor control mode 1251. In some variations, the controller may include physical buttons (e.g., buttons, pins, lever, etc.) on a housing of the controller. The user may press and/or touch one or more physical buttons to implement the motor control mode 1251.

For example, the user may press an “UP” button (e.g., on the user interface and/or on the controller) once to inflate an expandable member. For instance, in FIG. 12, the user may request that the expandable member be inflated. Consider that the user request includes a request to inflate the expandable member by x uL. In response to the user's request to inflate the expandable member by x uL, the controller may rotate the motor so that the plunger moves in a forward direction to deliver a desired amount of fluid (e.g., x uL in FIG. 12) to the expandable member. As discussed above, the circular gear may be positioned such that the gears are biased to move the plunger in the forward direction. Therefore, in response to the user's request to inflate, the controller may be configured to deliver the desired amount of fluid and wait for subsequent input from the user to proceed. To deliver the desired amount of fluid, the controller may transition smart-motor interface mode described in further detail below.

The user may press a “DOWN” button (e.g., on the user interface and/or on the controller) once to deflate the expandable member. For instance, in FIG. 12, the user may request that the expandable member be deflated by x uL. In response to the user's request to deflate the expandable member by x uL, the controller may be configured to implement the smart-motor interface mode 1253. More specifically, since the gears may be biased such that the plunger is geared towards the biasing linear direction (e.g., forward direction), the controller may be configured to determine a backlash compensation amount to re-align the gears so that they are positioned to gear the plunger in the backward direction. The controller may be configured to rotate the motor accordingly.

As discussed above, the controller may transition to implementing the smart-motor interface mode to inflate or deflate the expandable member by the desired amount. The smart-motor interface mode includes a move motor state 1243 that executes tasks to move the motor as desired, a wait motor state 1245 that executes tasks after the motor has completed a specific number of steps, and an idle state 1247 when the controller is neither delivering fluid to the expandable member nor withdrawing fluid from the expandable member.

As discussed above, the move motor state 1243 executes various steps required to move the motor to change a volume of the expandable member. For example, for a request to deflate the expandable member, in the move motor state 1243, the controller may check the settings of the motor (e.g., depicted as “check settings” in FIG. 12) to determine whether the motor is available for movement.

If the motor is available for movement, the controller may determine whether the request is to inflate or to deflate the expandable member. If the controller identifies that the request is to deflate the expandable member (e.g., for the plunger in the syringe to perform a withdrawal action), then the controller may implement “gear-loading”. When gear-loading is implemented, the controller may determine the backlash compensation amount to move the plunger in the backward direction. For example, as discussed above, the controller may access a lookup table with pre-determinedm amounts of backlash compensation for various syringe pump and/or expandable member pressures or pressure ranges. The controller may be configured to use this predetermined amount of backlash compensation to determine by how much to rotate the motor and/or gears to change a volume of the expandable member by the desired amount. In some variations, the controller may determine a total movement amount in the backward or proximal direction. The total movement amount may be a sum of the backlash compensation amount and the fluid transfer movement amount to deflate the expandable member by the desired amount. In some variations, the controller may additionally or alternatively determine an overall movement amount for one or more components of the syringe pump (e.g., gears, motor, plunger, etc.). The overall movement amount may include the total movement amount in the backward or proximal direction and a biasing movement amount in the distal or forward direction.

Therefore, if the controller is deflating the balloon, then the plunger may be performing a withdrawal action, and accordingly the controller may have to rotate the motor and/or gears to re-engage the gears (e.g., by the pre-determine amount of backlash compensation) and to deflate the expandable member by the desired amount. The controller may then have to rotate the motor and/or gears by the biasing movement amount in the opposite direction to re-engage the gears, during which the plunger may not materially move, or put differently, during which the plunger may remain substantially stationary. The plunger may then be geared for movement in the forward direction. Although the above steps have been described in relation to deflation, these steps may be applied a variation in which the controller may be inflating the expandable member.

In some variations, the controller may perform a safety check to determine the position and/or movement of the motor and/or other components of the syringe pump, such as the plunger. For example, the controller may receive data from a position sensor and/or a motion sensor (e.g., any of the sensors described herein such as an encoder, linear potentiometer, etc.) representative of a movement and/or position of the plunger, motor, or other component. The controller may then verify whether the current movement and/or position of the component is accurate. For example, the controller may retrieve data of the movement and/or position of the component at a previous time point. Based on the change of volume of the expandable member (e.g., inflations and deflation of the expandable member) from that previous time point to the current time point, the controller may calculate an estimated current movement and/or position of the component. The controller may compare the estimated current movement and/or position to the current movement and/or position obtained from the sensors. If the estimated current movement and/or position is within a threshold tolerance value of the current movement and/or position, then the controller may implement the “move motor” step.

For example, to implement the “move motor” step, the controller may execute the motor driver 1249 to move the motor and circular gear as required. To move the motor, the controller may first update the settings of the motor. That is, the settings may be updated based on the movement that may be needed for the motor and/or the gears to perform the user request. For example, as discussed above, if the controller is deflating the expandable member, then the plunger may be performing a withdrawal action. Therefore, the movement needed for the motor and/or gears to deflate the balloon may include additional steps of the motor and/or additional movement of the gears to compensate for backlash. The settings of the motor may be updated to include the additional movement for backlash. If, however, the controller is inflating the expandable member, then the plunger may be performing an injecting action. In variations in which the plunger is always geared towards performing the injection action (i.e., the gear(s) are biased), the motor and/or gears may not need additional movement to compensate for backlash. This may be updated in the settings of the motor.

After updating the settings, the controller may actuate the motor. The controller may also count the number of pulses for each step of motor actuation. For example, the number of pulses may be derived from a sensor such as a magnetic encoder. The total number of pulses may be indicative of the total number of steps that the motor may have taken. After counting the number of pulses, the controller may transition to the wait motor state 1245. In the wait motor state, the controller may perform another safety check such as described above to determine whether the position and/or movement of the motor or other components are accurate. If the position and/or movement of the motor or other components represents that the volume of the expandable member was changed by the intended amount and that the plunger moved in the intended direction, then the controller may implement the next step based on the direction in which the plunger moved. For example, if the position and/or movement of the motor represents that the expandable member was deflated and that the plunger moved in a backward direction, then the controller may move the motor by the biasing movement amount to re-engage the gears so that the plunger is geared towards the forward direction. In some variations, the biasing movement amount may be the same as the backlash compensation amount. The controller may move the gears such that the gears re-engage, thereby biasing the circular gear in a biasing rotational direction (e.g., the clockwise direction) and gearing the plunger towards the biasing linear direction (e.g., the forward direction). If, however, the position and/or movement of the motor represents that the expandable member was inflated and that the plunger moved in a forward direction, then the controller may transition to the idle state 1247.

In the idle state, the sensor (e.g., an encoder) may confirm whether the motor is moving. For instance, the sensor may determine the position of the motor every 10 seconds. If the change in the position is greater than a threshold value (e.g., change greater than 200 encoder ticks), this may be indicative of unwanted movement of fluid to or from the syringe pump (e.g., more than 10 microliters for a change greater than 200 encoder ticks). In such a scenario, the controller may trigger a warning signal to the user.

### Methods

Methods to compensate for backlash in syringe pumps are described herein. In some variations, the method may include biasing the gears in a biasing direction. In some variations, the method may not include biasing the gears in a biasing direction.

In variations in which the method include biasing the gears, in response to determining that the plunger is to be moved in a biasing linear direction by a desired amount, the method may include determining a fluid transfer movement amount to move the plunger by the desired amount. The method may include moving a component of the syringe pump (e.g., the motor, the circular gear, the linear gear, other gears in the gearbox, etc.)by the fluid transfer movement amount in the biasing direction. This may rotate the circular gear in the biasing rotational direction so as to move the plunger in the biasing linear direction by the desired amount. In response to determining that the plunger is to be moved in a direction opposite to the biasing linear direction by the desired amount, the method may include determining a backlash compensation amount and a fluid transfer movement amount. The method may include moving a component of the syringe pump (e.g., the motor, the circular gear, the linear gear, other gears in the gearbox, etc.) by the backlash compensation amount and by the fluid transfer movement amount in the direction opposite to the biasing direction. The method may then further include moving the component of the syringe pump by a biasing amount in the biasing direction.

For example, the method may include determining a total movement amount for the motor in a first direction (e.g., opposite the biasing direction). The total movement of the motor may include a combination of the backlash compensation amount and the fluid transfer movement amount in the first direction. In some variations, the method may include determining an overall movement amount for the motor. The overall movement amount may be the total movement amount in the first direction (opposite the biasing direction) and the biasing movement amount in a second, opposite direction (e.g., biasing direction). The method may include moving the motor by a sum of the backlash compensation amount and the fluid transfer movement amount (total movement amount in the first direction) such that the circular gear rotates in a direction counter to the biasing rotational direction, thereby moving the plunger in the direction opposite to the biasing linear direction by the desired amount. In some variations, the method may include determining the sum of the backlash compensation amount and the fluid transfer movement amount before moving the plunger in the direction opposite to the biasing linear direction by the desired amount. The method may further include moving the motor by the biasing movement amount to rotate the circular gear in the biasing rotation direction, thereby re-engaging the circular gear and the linear gear.

In variations in which the method does not include biasing the gears, the method may include determining whether the desired movement of the plunger is in the same direction as the preceding movement of the plunger. In response to determining that the desired movement of the plunger is in the same direction as the preceding movement of the plunger, the method may include determining a total movement of the motor which may be the same as the fluid transfer movement amount. The method may include moving the motor by the fluid transfer movement amount such that the circular gear rotate to move the plunger in the same direction as the preceding direction by the desired amount. In response to determining that the desired movement of the plunger is not in the same direction as the preceding movement of the plunger, the method may include determining a total movement for the motor, which may be the sum of the backlash compensation amount and the fluid transfer movement amount. The method may include moving the motor by the total movement amount such that the circular gear rotates to move the plunger in the desired direction.

The syringe pump described herein may be coupled to a blood flow control device for endovascular procedures. In variations in which the syringe pump is coupled to the blood flow control device, the method may include advancing an expandable member of a blood flow control device to a target location in a blood vessel of a patient. In response to determining that the expandable member is to be deflated (e.g., based on input received from a user and/or a determination made by the controller based on one or more blood pressure measurements), the method may include moving a component (e.g., motor, circular gear, linear gear, gears in the gearbox, plunger, etc.) of the syringe pump in a first direction (e.g., direction opposite to the biasing direction) to compensate for backlash in the syringe pump and to deflate the expandable member. After moving the component in the first direction, the method may include automatically moving the component in a second opposite direction (e.g., a biasing direction). In some variations, the expandable member of the blood flow control device may be advanced to the target location before compensating for backlash.

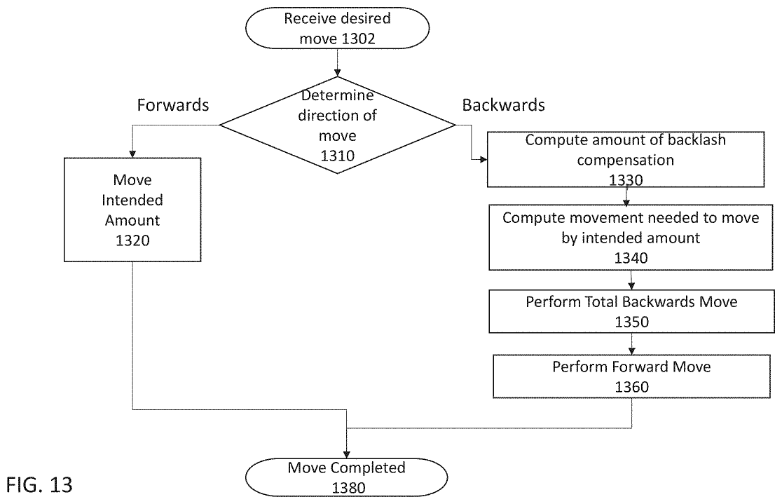

FIG. 13 is a flowchart illustrating an exemplary method of operating a syringe pump to compensate for backlash for variations in which the gears may be biased in a biasing direction. At 1302, the method may include determining via a processor (e.g., as part of a system controller, device controller, and/or pump controller) a desired movement for a motor of the syringe pump. The desired movement of the motor may be associated with an intended or desired movement of the plunger. For instance, if the intended movement of the plunger is in a biasing linear direction, then the desired movement of the motor and circular gear would be in a biasing rotational direction. If, however, the intended movement of the plunger is opposite the biasing linear direction, then the desired movement of the motor would be counter to the biasing rotational direction.

More specifically, if the intended movement of the plunger is in the forward direction, the desired movement of the motor may be a certain amount of rotational movement in a first rotational direction (e.g., clockwise direction). If the intended movement of the plunger is in the backward direction, the desired movement of the motor may be a certain amount of rotational movement in a second opposite rotational direction (e.g., counterclockwise direction).

To determine the desired movement of the motor, the method may include determining an amount of intended movement of the plunger. More specifically, the method may include determining an amount that the plunger is to be moved by. The method may include calculating a desired movement of the motor based on the amount of intended movement of the plunger. In some variations, as described above, calculating the desired movement of the motor may also include determining the pressure inside the cylindrical body of the syringe. In such variations, the desired movement may be based on the pressure. The desired movement of the motor may be a number of steps the motor may have to take to move the plunger by the amount of intended movement. For example, in variations in which the syringe pump described herein is coupled to an expandable member to change a volume of the expandable member, the intended amount of movement may be a bolus volume to change the volume of the expandable member (e.g., to inflate and/or deflate the expandable member). The method may include determining the number of steps the motor may have to take to move the bolus volume from the syringe pump (e.g., for deflation of the expandable member) or to the syringe pump (e.g., for inflation of the expandable member).

At 1310, the method may identify the direction of the desired movement of the plunger. If the direction of the intended movement of the plunger is forward, then at 1320, the method may include rotating the motor (or the circular gear) in the first rotational direction by the desired movement. For example, the method may include setting a drive current of the motor based on the desired movement of the motor calculated at 1302. For instance, the method may include setting a drive current of the motor to move the motor by the determined number of steps based on the bolus volume calculated at 1302 and the direction of the intended movement. The method may also include receiving from a sensor (e.g., optical encoder, magnetic encoder, contact sensor, and/or the like) a first position and/or movement of the motor or other components such as plunger. This first position and/or movement may be representative of the position and/or movement before the motor has been rotated by the desired movement. The method may include controlling the motor (e.g., via the controller) to rotate by the desired movement (e.g., number of steps needed for the intended movement of the plunger). The method may then include receiving from the sensor a second position and/or movement of the motor or other components, computing a difference between the second position and/or movement and the first position and/or movement. This difference may correspond with desired movement of the motor. This difference may be compared to an expected movement of the motor. If the error between the difference and the expected movement of the motor is greater than a threshold, then the method may include transmitting the error (e.g., via a user interface) to the user. If not, the method may include transitioning to an idle motor state such as described above.

If however, the direction of the desired movement of the plunger is backward, then at 1330, the method may include determining an amount of backlash compensation. Since the motor is biased such that the circular gear is biased to rotate in the clockwise direction gearing the plunger in the forward direction, to move the plunger in the backward direction backlash may need to be compensated for. Accordingly, the method may include calculating a backlash compensation amount. As discussed above, the backlash compensation amount may include the number of additional steps of the motor that may be needed to re-engage the linear gear and the circular gear. In some variations, the backlash compensation amount may be based on a lookup table. In some variations, the backlash compensation amount may be calculated based on a pressure inside a cylindrical body of the syringe.

At 1340, the method may include calculating a movement of the motor to move the plunger by the desired amount. As discussed at 1302, a movement of the motor may be calculated based on a number of steps that the motor may need to remove and/or fill the syringe pump with the bolus volume (e.g., deflate and/or inflate the expandable member) and the direction of the desired movement of the plunger. Therefore, the desired movement of the motor calculated in 1302 may include the number of steps for a deflation (e.g., caused to the expandable member by movement of plunger in the backward direction) bolus volume.

At 1350, the method may include rotating the motor (or the circular gear) in the second opposite rotational direction by a total movement of the motor. In some variations, the method may include calculating the total desired movement of the motor. The total desired movement of the motor may be a sum of the amount of backlash compensation calculated at 1330 and the movement of the motor to move the plunger by the intended amount calculated at 1340. After calculating the total desired movement of the motor, in some variations, the method may include setting a drive current of the motor based on the total desired movement of the motor calculated at 1350. For instance, the method may include setting a drive current of the motor to move the motor by the determined number of steps as calculated at 1350. The method may also include receiving from a sensor (e.g., optical encoder, magnetic encoder, contact sensor, and/or the like) a first position and/or movement of the motor or other components such as plunger. This first position and/or movement may be representative of the position and/or movement before the motor has been rotated by the total desired movement. The method may then include controlling the motor (e.g., via the controller) to rotate by the total desired movement (e.g., total number of steps needed for backlash compensation and for the intended movement of the plunger) in a second opposite rotational direction.

In some variations, the method may also include receiving from the sensor a second position and/or movement of the motor or other components, computing a difference between the second position and/or movement and the first position and/or movement. This difference may correspond with the total desired movement of the motor. This difference may be compared to an expected movement of the motor. If the error between the difference and the expected movement of the motor is greater than a threshold, then the method may include transmitting the error (e.g., via a user interface) to the user.

At 1360, the method may include rotating the motor (or the circular gear) in the first rotational direction based on the biasing movement amount, which may, in some variations, be the same as the backlash compensation amount determined at 1330. The method may then include receiving from the sensor a third position and/or movement of the motor or other component, computing a difference between the third position and/or movement and the second position and/or movement. This difference may correspond with the total movement amount of the motor. This difference may be compared to an expected movement of the motor. If the error between the difference and the expected movement of the motor is greater than a threshold, then the method may include transmitting the error (e.g., via a user interface) to the user. If not, the method may include transitioning to an idle motor state such as described above.

### Systems

### Exemplary Blood Flow Control System

The syringe pumps described herein may be incorporated into a blood flow control system. The blood flow control system may include an expandable member that may be strategically placed in a blood vessel of a patient. The blood flow control system may automatically control a volume of the expandable member to control blood flow in the blood vessel. In some variations, the blood flow control system may deliver precise amount of fluid (e.g., medications, blood products, etc.) to a patient.

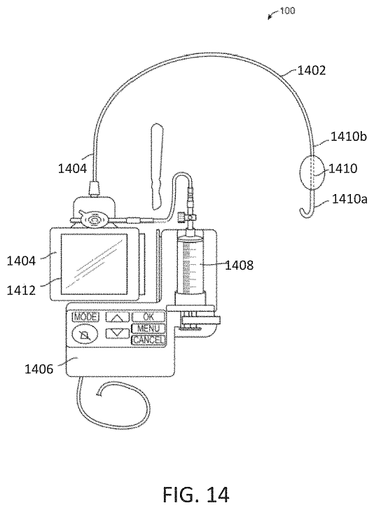

FIG. 14 illustrates an exemplary variation of a blood flow control system 1400. The blood flow control system 1400 may comprise a blood flow control device 1404 and a system controller 1416. The blood flow control device may comprise an elongate body 1402, such as, for example, a catheter, an expandable member 1410, such as, for example, a balloon, and one or more controllers, such as, a device controller 1412, which may comprise a user interface. The blood flow control device 1404 may additionally comprise or otherwise be operably coupled to a pump 1408 (which may be any of the syringe pumps described herein). The expandable member 1410 may be disposed on, coupled to, integrated with, attached to, or otherwise affixed to the elongate body 1402. The blood flow control device 1404 may also comprise one or more sensors (not shown in FIG. 14). In variations in which the blood flow control device 1404 comprises one or more sensors, the sensors may be disposed on, coupled to, integrated with, attached to, or otherwise affixed to the elongate body 1402 and/or may be positioned on or within the device and/or system controllers. In other variations, one or more of the sensors may be external to or separate from the blood flow control device 1404. The sensor(s) may be operably coupled to one or both of the device controller 1412 and the system controller 1406. In some variations, the sensor(s) and thereby the blood flow control device 1404 may be communicably coupled to the system controller 1406 (e.g., via the device controller 1412). The expandable member 1410 may be fluidly coupled to the pump 1408. As described in more detail herein, the pump 1408 may be a syringe pump comprising a syringe and optionally, a pump controller. For example, in some variations, the pump 1408 may comprise its own controller (pump controller) as described in more detail herein, while in other variations, the pump 1408 may be controlled by the device and/or system controllers. The controller for the pump (e.g., pump controller, device controller, system controller) may include a motor (not shown in FIG. 14), and the motor may be controlled by any of the aforementioned controllers. Moreover, while the device controller 1412 and the system controller 1416 are described above as two controllers, it should be appreciated that a single controller could be utilized to perform the functions of both the device controller 1412 and the system controller 1416 described herein, and/or any of the functions of the device controller 1412 could be performed by the system controller 1416 and vice versa. Moreover, as noted above, the single controller could additionally be utilized to perform the functions of the pump controller described herein, and/or any of the functions of the pump controller could be performed by either or both of the device controller 1412 and the system controller 1416. Accordingly, any of the components described herein as coupled to either the device controller 1412, the system controller 1416, or the pump controller may be coupled to the other of the device controller 1412, the system controller 1416, the pump controller or to more than one of the controllers, as the case may be. In variations in which the pump controller is separate from the device and/or system controller 1412, 1416, the pump controller described may be communicatively coupled to one or more of the device controller 1412 and the system controller 1416. In other variations, the pump controller and may form part of the device controller 1412 and/or the system controller 1416.

### Blood Flow Control Device

As described above and depicted in FIG. 14, the blood flow control device 1404 may comprise an elongate body 1402, an expandable member 1410 coupled to the elongate body 1402, and one or more sensors coupled to or integrated with a shaft of the elongate body 1402.

### Elongate Body

The elongate body 1402 may comprise a shaft sized and shaped for placement within a blood vessel (e.g., aorta, vein, etc.) of a patient. In some variations, the elongate body 1402 may have a length sufficient to reach a patient's aorta via the femoral or radial artery. For example, in some variations, the elongate body 1402 may be a catheter configured to be inserted into the femoral or radial artery and to extend through the patient's vasculature into the aorta. In some variations, the elongate body 1402 may be steerable. For example, in some variations, the elongate body 1402 be mechanically coupled to knobs, levers, pullwires, and/or the like that may be used to steer or otherwise deflect a distal end of the shaft of the elongate body 1402. In some variations, the elongate body 1402 may include one or more lumens (not shown in FIG. 14) therethrough. The lumen(s) may be partial lumen(s) (e.g., open on one end) and may be disposed within or lie within the movable shaft. Alternatively, the movable shaft may define one or more lumen(s). In some variations, the lumen(s) may include an intake or inflation lumen and an exhaust or deflation lumen to deliver fluid and/or compressed gas to the expandable member and to recover the fluid and/or compressed gas from the expandable member 1410, respectively.

### Expandable Member

The expandable member 1410 may be one of disposed on, coupled to, integrated with, attached to, and/or affixed to the shaft of the elongate body 1402 and a size of the expandable member may be controllable by a controller or a user. For example, the expandable member may be configured to expand and contract and/or inflate and deflate such that the size (e.g., volume) of the expandable member may change during use of the blood flow control system. In some variations, the expandable member may be an inflatable/deflatable balloon, while in other variations the expandable member may comprise a shape memory material, in yet other variations, the expandable member may be connected to mechanical linkage (e.g., wires, etc.) to change the size of the expandable member. The expandable member 1410 may comprise any suitable elastomeric material (e.g., polyurethane, silicone, etc.). Alternatively, the expandable member 1410 may comprise polyester, nylon, etc. During use, blood flow may be regulated or otherwise controlled by changing a size of the expandable member 1410, thereby altering the area of the blood vessel that is occluded by the expandable member 1410. Fluid and/or compressed gas may be delivered through one or more lumens in the elongate body 1402 in order to control and/or adjust the size (e.g., volume) of the expandable member 1410. Thus, in some variations, the expandable member 1410 may be strategically placed within the aorta of a patient and the size of the expandable member 1410 may control blood flow through the aorta of the patient such that blood flow distal to expandable member 1410 may be impeded to augment blood pressure proximal to expandable member 1410. The outer surface of the expandable member 1410 may be configured to contact or otherwise interface with the wall(s) of the patient's blood vessel (e.g., at complete occlusion).

Although FIG. 14 illustrates one expandable member 1410 configured to regulate blood flow through the aorta of the patient, it should be readily understood that the blood flow control device 1404 may include any number of suitable expandable members 1410. For instance, the blood flow control device 1404 may include two expandable members 1410 disposed on, coupled to, integrated with, attached to, and/or affixed to the elongate body 1402 in series. Similarly, the blood flow control device may include three expandable members disposed on, coupled to, integrated with, attached to, and/or affixed to the elongate body 1402 in series spaced at equal distance from each other. In some variations, the expandable member 1410 may comprise a plurality of balloons (e.g., two, three, four, or more) positioned in series along the length of the elongate body 1402 or disposed within each other. In variations comprising a plurality of balloons, the balloons may be configured to expand and contract individually or separately.

### Sensor(s)

The blood flow control system may comprise one or more sensors (e.g., two, three, four, five, or more). In some variations, the blood flow control device may itself comprise one or more sensors, while in other variations, one or more sensors may be integrated into the system separately from the blood flow control device. For example, one or more sensors (e.g., a distal sensor, a proximal sensor) may be integrated with and/or disposed on the elongate body 1402 of the blood flow control device. For example, the sensors may be integrated into the elongate body 1402 in a manner similar to that described in U.S. Provisional Application No. 63/277,428, the content of which is hereby incorporated by reference in its entirety.

Additionally or alternatively, one or more sensors may be disposed on tubing that may be coupled to open ports in elongate body 1402. For example, one or more sensors (e.g., a proximal sensor, a distal sensor) may be connected via a saline-filled tube that may connect to open ports that are proximal or distal to the expandable member 1410. Put differently, instead of being disposed on the elongate body 1402, these sensors may be coupled to saline-filled tubes that are fluidly coupled to the elongate body 1402 (e.g., at ports proximal or distal to the expandable member) via the saline-filled tube. In such variations, the pressure along the saline-filled tube may be measured by the proximal sensor and the distal sensor.

In yet other alternative variations, the one or more sensors may be integrated into and/or disposed on the blood flow control system 1400 via a combination of the saline-filled tube and via one or more wires.

In variations in which the blood flow control device comprises one or more sensors, the blood flow control device may comprise any suitable number of sensors (e.g., two, three, four, five, or more) and the sensors may be positioned in any suitable location for measuring a physiologic condition of the patient and/or a characteristic of the expandable member. For example, the blood flow control device may comprise a first, distal sensor, and a second, proximal sensor. The distal sensor, the position of which is indicated by reference numeral 1410b, may be disposed between a tip of the elongate body 1402 and the expandable member 1410. A proximal sensor, the position of which is indicated by reference numeral 1410a, may be disposed between the base of the elongate body 1402 (where the elongate body 1402 couples to device controller 1412) and the expandable member 1410. Each of the distal sensor and the proximal sensor may measure patient physiologic information, such as physiologic information indicative of blood flow through and/or blood pressure within a blood vessel (e.g., the aorta), to determine the patient's underlying physiology. For example, the distal sensor and the proximal sensor may measure a local blood pressure of the patient at or around the position of the respective sensor. For example, the distal sensor may measure a blood pressure of the patient within the blood vessel at a region surrounding 1410b and the proximal sensor may measure a blood pressure of the patient within the blood vessel at a region surrounding 1410a. The data from the distal sensor may be used to measure the distal systolic pressure and the distal diastolic pressure of the patient. For instance, distal systolic pressure and distal diastolic pressure may be inferred from a waveform of the blood pressure. Distal systolic pressure may be measured by analyzing peaks of the waveform for a given time duration. Distal diastolic pressure may be measured by analyzing valleys of the waveform for the given time duration. Distal mean arterial pressure may be measured from the distal systolic pressure and the distal diastolic pressure. In a similar manner, the data from the proximal sensor may be used to measure the proximal systolic pressure and the proximal diastolic pressure of the patient. For instance, proximal systolic pressure and proximal diastolic pressure may be inferred from a waveform of the blood pressure. Proximal systolic pressure may be measured by analyzing peaks of the waveform for a given time duration. Proximal diastolic pressure may be measured by analyzing valleys of the waveform for the given time duration. Proximal mean arterial pressure may be measured from the proximal systolic pressure and the proximal diastolic pressure.

Although the proximal sensor and the distal sensor may measure a blood pressure of the patient, in some variations, the blood pressure may be used to calculate one or more of heart rate, respiratory rate, blood flow rate, cardiac output of the patient, and/or the like.

Note that the terms “proximal” and “distal,” as used herein in relation to sensor(s) and/or particular localized blood pressure readings, refer to blood flow directionality from the heart. That is, “proximal” is closer to the heart while “distal” is further from the heart. This is not to be confused with the reversed usage of the terms when described from the perspective of a medical device such as a catheter, where the “distal end” of the medical device would commonly be understood as the end with the expandable element 1410 furthest from the system controller 106 and the “proximal end” would be understood as the end closer to the operator.

In some variations, the blood flow control device may further comprise an expandable member sensor (not shown in FIG. 14). In some variations, the expandable member sensor may be coupled to, integrated with and/or disposed on the expandable member 1410 or on the elongate body 1402 within the expandable member 1410. In some variations, the expandable member sensor may be coupled to, integrated with and/or disposed on the device controller 1412 or the system controller 1406, and may be fluidly coupled to the expandable member.

The expandable member sensor may detect a characteristic of the expandable member, such as, for example, a pressure of fluid and/or compressed gas inside the expandable member 1410. In some variations, the pressure and/or changes to the pressure of fluid and/or compressed gas inside the expandable member 1410 may be analyzed to detect one or more errors. For instance, the expandable member sensor may detect when the pressure of the fluid and/or compressed gas inside the expandable member 1410 is too high. In some variations, the expandable member sensor may detect an unexpected pressure change inside the expandable member 1410. This may be indicative of a rupture in the expandable member 1410. In some variations, if the trend of the change in pressure inside the expandable member 1410 is dissimilar with the expected change based on the trend in proximal pressure or distal pressure, the expandable member sensor may detect this difference from expected change. In some variations, the expandable member sensor may detect spikes in the pressure inside the expandable member 1410 during changes to the movement of the pump 1408 in order to detect if the movement of the pump 1408 corresponds to the expected pressure inside the expandable member 1410. In some variations, the expandable member sensor may detect the amount (e.g., a volume) of fluid and/or compressed gas that has been added or removed from the expandable member 1410.

Additionally or alternatively, in some variations, the blood flow control device may further optionally comprise a flow sensor (not shown in FIG. 14). The flow sensor may be integrated with and/or disposed on the expandable member 1410 and may measure the amount and/or rate of blood flowing past the expandable member 1410. In variations in which a blood flow sensor is not included, the amount and/or rate of blood flowing past the expandable member 1410 may be determined from measurements obtained from other sensors, such as, for example, one or more of the proximal sensor, the distal sensor, and the expandable member sensor.

In some variations, the blood flow control device may further comprise a barometer (not shown in FIG. 14). The barometer may be integrated with and/or disposed within a housing of the device controller 1412 and/or may be disposed within the elongate body 102 and may be communicatively coupled to the device controller 1412. In some variations, the barometer may be integrated with and/or disposed within a housing of the system controller 1406 and may communicatively couple thereto. The system may also comprise a plurality of barometers, such as, for example, device controller barometer and a system controller barometer. The one or more barometers may measure ambient pressure at the location of the patient. For instance, the proximal sensor and the distal sensor may measure the absolute blood pressure. However, the barometer may measure the ambient pressure at the location of the patient. Accordingly, the blood pressure reported by the blood flow control system 1400 may be blood pressure that is relative to the ambient pressure at the location of the patient (e.g., taking into consideration changes to ambient pressure as the patient is transported). Additionally or alternatively, the blood flow control device may include a gauge sensor to measure the relative pressure of the blood relative to the ambient air.

### Device Controller

The blood flow control device 1404 may comprise a device controller 1412, which may be coupled to a base of the elongate body 1402. The device controller 1412 may be communicatively coupled to one or more sensors, such as, for example, the proximal sensor, the distal sensor, and/or the expandable member sensor. For example, the device controller 1412 may be electronically coupled to the proximal sensor, the distal sensor and/or the expandable member sensor.

In some variations, the device controller 1412 may comprise a housing. The housing may be coupled to the elongate body 102 and may contain a number of electronic components, such as, for example, a biasing circuit, an optional amplifier, a filter, and an Analog-to-Digital Conversion (ADC) circuit. The ADC circuit may output the readings obtained from the sensors (e.g., the proximal sensor and the distal sensor), thereby indicating a physiologic condition of the patient. For example, in some variations, the proximal sensor and the distal sensor may each include three connection wires—a power wire and two output wires. The output wires may be connected to the biasing circuit and the power wire. The biasing circuit may provide power to the power wire and appropriate resistance to the two output wires. The two output wires may be coupled to the amplifier which may amplify the differential voltage created across the two output wires. The amplifier may be coupled to the filter which may reduce high frequency and/or low frequency noise from the output of the amplifier. The output of the filter may be coupled to the Analog-to-Digital Conversion (ADC). The output of the ADC may be at a variety of rates and sample sizes indicating a physiologic condition of the patient. The device controller 1412 may include any of the components and/or features described with respect to the system controller 106 described herein. In some variations, the device controller 1412 may be used primarily as a user interface (e.g., it may not provide a controlling capability for the blood flow control device).

### System Controller

In some variations, the blood flow control system may comprise a system controller 106 in addition to the device controller 1412. The system controller 106 may be coupled to the blood flow control device 1404, for example, via the device controller 1412, or in variations without a device controller 1412, via the elongate body 1402 directly. The device controller 1412 may be communicably coupled to the sensors in the system. For example, the system controller 1406 may be communicably coupled to one or more of the proximal sensor, the distal sensor, the expandable member sensor, the barometer, and the flow sensor (when included), and, in variations comprising more than one controller, may be communicably coupled to the device controller 1412. For example, in some variations, the proximal sensor and the distal sensor may be electronically coupled to the device controller 1412, which in turn may be communicably coupled to the system controller 1406.

The device controller 1412 and/or the system controller 1406 may comprise a housing, which may contain a number of electronic components, such as, for example, the biasing circuit, the amplifier, the filter, and the ADC circuit. The sensor readings extracted from the ADC circuit may be transmitted from the device controller 1412/system controller 1406 to the system controller 1406/device controller 1414.

In some variations, the device controller 1412 and/or the system controller 1406 may further comprise a motion sensor and/or a position sensor communicably coupled to the pump 1408. In some variations, the position sensor may measure a position of a portion of the pump 1408. For instance, the position sensor may measure a position of a plunger of a syringe pump 1408. The position of the portion of the pump 1408 may be used to infer the amount of fluid that has been delivered to and/or removed from the expandable member 1410.

Additionally or alternatively, the system 1406 and/or device 1412 controller may comprise a motion sensor and/or position sensor etc.) to measure a position and/or motion of another component of the system, such as, for example, an actuation mechanism for the pump (e.g., a motor configured to actuate the syringe), the actuator (e.g., circular gear), or another gear or component in the system. In some variations, the motion sensors may include encoders, such as, for example, magnetic encoders, optical encoders, etc. In variations comprising encoders, the encoders may monitor movement of the component, e.g., the motor, the circular gear, the linear gear or other portion of the plunger, etc., and the movement may be used to control actuation of the syringe and/or to determine the amount of inflation and/or deflation in the expandable member. More specifically, for example, if the syringe is actuated using a motor, the encoder may monitor movement of the motor, and the motor's movement may be used to determine the amount of inflation and/or deflation in the expandable member 1410 and/or may be used to control actuation of the syringe. In some variations, the flow sensor described above may determine the amount of inflation and/or deflation in the expandable member 1410.

Alternatively, the proximal sensor and the distal sensor may be electronically coupled to the system controller 1406. The system controller 106 may comprise a housing. The housing may contain a number of electronic components, such as, for example, a biasing circuit, an amplifier, a filter, and an Analog-to-Digital Conversion (ADC) circuit. The ADC circuit may output the readings obtained from the sensors (e.g., the proximal sensor and the distal sensor), thereby indicating a physiologic condition of the patient. For example, in some variations, the proximal sensor and the distal sensor may each include three connection wires — a power wire and two output wires. The output wires may be connected to the biasing circuit and the power wire. The biasing circuit may provide power to the power wire and appropriate resistance to the two output wires. The two output wires may be coupled to the amplifier which may amplify the differential voltage created across the two output wires. The amplifier may be coupled to the filter which may reduce high frequency and/or low frequency noise from the output of the amplifier. The output of the filter may be coupled to the Analog-to-Digital Conversion (ADC). The output of the ADC may be at a variety of rates and sample sizes indicating a physiologic condition of the patient.

Accordingly, the sensor readings from the proximal sensor and the distal sensor may be extracted directly at the system controller 1406. In some variations, the expandable member pressure sensor, the barometer, and optionally the flow sensor may transmit sensor data directly to the system controller 1406. The data from the sensors in the system may be collected continuously or intermittently and may be collected over a defined period of time. In some variations, the data from the proximal sensor and the distal sensor may be collected continuously, such as for example, every 3 seconds, 4 seconds, 5 seconds, 6 seconds, 7 seconds, 8 seconds, 9 seconds, or 10 seconds (including all values and sub-ranges therein, such as, for example, between about 3 second and about 6 second, about 4 second and about 6 second, or between about 5 second or about 6 second). In some variations, the data from the proximal sensor and the distal sensor may be collected every 5 seconds at 200 Hz.

In some variations, data may be collected from the expandable member sensor continuously such as, for example, every 3 seconds, 4 seconds, 5 seconds, 6 seconds, 7 seconds, 8 seconds, 9 seconds, or 10 seconds (including all values and sub-ranges therein, such as, for example, between about 3 second and about 6 second, about 4 second and about 6 second, or between about 5 second or about 6 second).

In some variations, data from the sensors may be analyzed over a discrete period of time. For instance, the data may be analyzed for example, every 3 seconds, 4 seconds, 5 seconds, 6 seconds, 7 seconds, 8 seconds, 9 seconds, or 10 seconds (including all values and sub-ranges therein, such as, for example, between about 3 second and about 6 second, about 4 second and about 6 second, or between about 5 second or about 6 second). In some variations, the pump 108 may include a stepper motor. In such variations, the data may be analyzed based on the motion of the stepper motor (e.g., 1300 steps per second) and/or based on the sequence of the movement of the motor (e.g., between 25-2000 milliseconds).

The system controller 1406 may include one or more processors (e.g., CPU). The processor(s) may be any suitable processing device configured to run and/or execute a set of instructions or code, and may include one or more data processors, image processors, graphics processing units, digital signal processors, and/or central processing units. The processor(s) may be, for example, a general purpose processor, a Field Programmable Gate Array (FPGA), an Application Specific Integrated Circuit (ASIC), and/or the like. The processor(s) may be configured to run and/or execute application processes and/or other modules, processes and/or functions associated with the blood flow control system 1400.

In some variations, the system controller 1406 may run and/or execute application processes and/or other modules. These processes and/or modules when executed by a processor may be configured to perform a specific task. These specific tasks may collectively enable the system controller 1406 to automatically operate and control the blood flow control system 1400 while detecting errors and responding to the errors. Specifically, these specific tasks may enable the system controller 1406 to detect errors and automatically adjust inflation and/or deflation of the expandable member 1410 accordingly.

The system controller 1406 may comprise a processor. Generally, the processor (e.g., CPU) described here may process data and/or other signals to control one or more components of the system. The processor may be configured to receive, process, compile, compute, store, access, read, write, and/or transmit data and/or other signals. In some variations, the processor may be configured to access or receive data and/or other signals from one or more of a sensor (e.g., proximal sensor, distal sensor, expandable member sensor, etc.) and a storage medium (e.g., memory, flash drive, memory card). In some variations, the processor may be any suitable processing device configured to run and/or execute a set of instructions or code and may include one or more data processors, image processors, graphics processing units (GPU), physics processing units, digital signal processors (DSP), analog signal processors, mixed-signal processors, machine learning processors, deep learning processors, finite state machines (FSM), compression processors (e.g., data compression to reduce data rate and/or memory requirements), encryption processors (e.g., for secure wireless data and/or power transfer), and/or central processing units (CPU). The processor may be, for example, a general-purpose processor, Field Programmable Gate Array (FPGA), an Application Specific Integrated Circuit (ASIC), a processor board, and/or the like. The processor may be configured to run and/or execute application processes and/or other modules, processes and/or functions associated with the system. The underlying device technologies may be provided in a variety of component types (e.g., metal-oxide semiconductor field-effect transistor (MOSFET) technologies like complementary metal-oxide semiconductor (CMOS), bipolar technologies like generative adversarial network (GAN), polymer technologies (e.g., silicon-conjugated polymer and metal-conjugated polymer-metal structures), mixed analog and digital, and/or the like.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

Generally, the blood flow control systems described here may comprise a memory configured to store data and/or information. In some variations, the memory may comprise one or more of a random access memory (RAM), static RAM (SRAM), dynamic RAM (DRAM), a memory buffer, an erasable programmable read-only memory (EPROM), an electrically erasable read-only memory (EEPROM), a read-only memory (ROM), flash memory, volatile memory, non-volatile memory, combinations thereof, and the like. In some variations, the memory may store instructions to cause the processor to execute modules, processes, and/or functions associated with a blood flow control device, such as signal waveform generation, expandable element control, data and/or signal transmission, data and/or signal reception, and/or communication. Some variations described herein may relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes. Examples of non-transitory computer-readable media include, but are not limited to, flash memory, non-volatile memory (e.g., Intel® OptaneTM), magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical discs; solid state storage devices such as a solid state drive (SSD) and a solid state hybrid drive (SSHD); carrier wave signal processing modules; and hardware devices that are specially configured to store and execute program code such as Application-Specific Integrated Circuits (ASICs), Programmable Logic Devices (PLDs), Read-Only Memory (ROM), and Random-Access Memory (RAM) devices. In some variations, the system controller 1406, the device controller 1412, and/or the pump controller may be integrated into a single controller.

### Communication Device or Module

In some variations, the system controller 1406 may include at least one communication device or module, such as a wireless communication module to communicate with one or more other devices. For example, the communication module may be configured to communicate data (e.g., sensor data, target blood pressures, target blood pressure ranges, state of the blood flow control system, such as internal temperature of blood flow control system, battery charge level of the blood flow control system, time of day, and/or properties of the blood flow control system, such as hardware and firmware revision number of the blood flow control system, system capabilities, etc.) and/or determinations or calculations made based on the data (e.g. errors, physiologic states, clinical decision support), to one or more devices, such as, for example, an external computer, a mobile device (e.g., a smartphone), a tablet, or the like. The communication device may comprise a network interface configured to connect the blood flow control device to another device or system (e.g., Internet, remote server, database) by wired or wireless connection. In some variations, the blood flow control device and/or system may be in communication with other devices (e.g., cell phone, tablet, computer, smart watch, and the like) via one or more wired and/or wireless networks. In some variations, the network interface may comprise one or more of a radiofrequency receiver/transmitter, an optical (e.g., infrared) receiver/transmitter, and the like, configured to communicate with one or more devices and/or networks. The network interface may communicate by wires and/or wirelessly with one or more of the blood flow control device, system controller 106, network, database, and server.

The network interface may comprise RF circuitry configured to receive and/or transmit RF signals. The RF circuitry may convert electrical signals to/from electromagnetic signals and communicate with communication networks and other communication devices via the electromagnetic signals. The RF circuitry may comprise well-known circuitry for performing these functions, including but not limited to an antenna system, an RF transceiver, one or more amplifiers, a tuner, one or more oscillators, a mixer, a digital signal processor, a CODEC chipset, a subscriber identity module (SIM) card, memory, and so forth.

Wireless communication through any of the devices may use any of plurality of communication standards, protocols and technologies, including but not limited to, Global System for Mobile Communication (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11b, IEEE 802.11g, IEEE 802.11n, and the like), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol. In some variations, the devices herein may directly communicate with each other without transmitting data through a network (e.g., through NFC, Bluetooth, Wi-Fi, RFID, and the like).

The communication device or module may include a wireless transceiver that is integrated into the system controller 106. However, the blood flow control system may additionally or alternatively include a communication module that is separate from the system controller 106.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A method of operating a syringe pump comprising a syringe, a processor communicably coupled to the syringe, a first gear and a second gear, the method comprising:
determining, via the processor, a backlash compensation amount;
determining, via the processor, a fluid transfer movement amount;
moving, via the processor, at least one component of the syringe pump the backlash compensation amount and the fluid transfer movement amount in a first direction; and
moving, via the processor, the at least one component of the syringe pump a biasing movement amount in a second direction opposite the first direction to re-engage the first gear and the second gear.

2. The method of claim 1, wherein an absolute value of the biasing movement amount is equal to an absolute value of the backlash compensation amount.

3. The method of claim 1, wherein moving the at least one component comprises moving a motor operably coupled to the first gear.

4. The method of claim 1, wherein moving the at least one component comprises moving the first gear.

5. The method of claim 1, wherein the first gear is a circular gear and the second gear is a linear gear.

6. The method of claim 5, wherein a plunger of the syringe comprises the linear gear.

7. The method of claim 1, wherein the syringe pump comprises a gearbox operably coupling a motor to the first gear, and wherein the backlash compensation amount is based at least in part on a gear ratio of the gearbox.

8. (canceled)

9. The method of claim 7, wherein the gear ratio of the gearbox is between about 40:1 and about 150:1.

10. (canceled)

11. The method of claim 1, wherein the method further comprises receiving, at the processor, a pressure measurement indicative of a pressure inside the syringe and wherein the backlash compensation amount is based at least in part on the pressure measurement.

12. (canceled)

13. The method of claim 1, wherein the first direction is a fluid withdrawal direction and the second direction is a fluid injection direction.

14-16. (Canceled)

17. The method of claim 1, further comprising determining a sum of the backlash compensation amount and the fluid transfer movement amount, and wherein the sum is determined before moving the at least one component in the first direction.

18. (canceled)

19. The method of claim 1, wherein the syringe pump comprises a plunger including a linear gear, and a controller comprising a circular gear, wherein the circular gear is operably coupled to the linear gear, and wherein moving the at least one component in the second direction re-engages the linear gear with the circular gear without materially moving the plunger.

20. A syringe pump comprising:
a cylindrical body;
a plunger partially disposed within the cylindrical body, wherein the plunger comprises a first gear; and
a controller comprising a second gear operably coupled to the first gear wherein the is controller is configured to:
determine a backlash compensation amount;
determine a fluid transfer movement amount;
move at least one component of the syringe pump the backlash compensation amount and the fluid transfer movement amount in a first direction; and
move the at least one component of the syringe pump a biasing movement amount in a second direction opposite the first direction to re-engage the first gear and the second gear.

21. The syringe pump of claim 20, wherein an absolute value of the biasing movement amount is equal to an absolute value of the backlash compensation amount.

22. The syringe pump of claim 20, wherein the controller further comprises a motor operably coupled to the second gear to actuate the second gear, and wherein the at least one component includes the motor.

23. The syringe pump of claim 20, wherein the first gear is a linear gear and the second gear is a circular gear, and wherein the at least one component includes the circular gear.

24. The syringe pump of claim 20, wherein the controller further comprises a gearbox operably coupled to the second gear, and wherein the backlash compensation amount is based at least in part on a gear ratio of the gearbox.

25. (canceled)

26. The syringe pump of claim 24, wherein the gear ratio of the gearbox is between about 40:1 and about 150:1.

27. (canceled)

28. The syringe pump of claim 20, wherein the controller further comprises a pressure sensor configured to provide a pressure measurement indicative of a pressure inside the cylindrical body, and wherein the backlash compensation amount is based at least in part on the pressure inside the cylindrical body.

29. (canceled)

30. The syringe pump of claim 20, wherein the first direction is a fluid withdrawal direction and the second direction is a fluid injection direction.

31. A blood flow control system comprising:
the syringe pump of claim 20; and
a blood flow control device fluidly coupled to the syringe pump.

32. (canceled)

33. A syringe pump comprising:
a cylindrical body;
a plunger partially disposed within the cylindrical body, wherein the plunger comprises a linear gear; and
a controller comprising a circular gear operably coupled to the linear gear, a motor operably coupled to the circular gear to actuate the circular gear, and a processor communicably coupled to the motor, wherein the processor is configured to bias the circular gear in a biasing rotational direction after the syringe pump transfers fluid.

34. The syringe pump of claim 33, wherein biasing the circular gear in the biasing rotational direction positions the plunger to move in a biasing translational direction.

35. The syringe pump of claim 33, wherein the syringe pump is configured such that the plunger remains substantially stationary when the circular gear is biased in the biasing rotational direction.

36. (canceled)

37. (canceled)

38. The syringe pump of claim 33, wherein the processor is configured to automatically bias the circular gear after the syringe pump receives fluid.

39. The syringe pump of claim 33, wherein the processor is configured to automatically bias the circular gear after the syringe pump delivers fluid.

40. (canceled)

41. A method comprising:
advancing an expandable member of a blood flow control device to a target location in a blood vessel of a patient, wherein the expandable member is fluidly coupled to a syringe pump;
in response to determining that the expandable member is to be deflated, moving a component of the syringe pump in a first direction to compensate for backlash in the syringe pump and to deflate the expandable member; and
after moving the component in the first direction, automatically moving the component in a second opposite direction.

42. The method of claim 41, where moving the component of the syringe pump in the first direction deflates the expandable member.

43-46. (canceled)

47. The method of claim 41, wherein the syringe pump comprises a plunger, and wherein moving the component in the first direction causes fluid transfer.

48. The method of claim 41, wherein the syringe pump comprises a plunger including a linear gear, and a controller comprising a circular gear, wherein the circular gear is operably coupled to the linear gear, and wherein moving the component in the second opposite direction re-engages the linear gear with the circular gear without materially moving the plunger.

49-96. (canceled)
